SEQUENCE LISTING (30 sequences)

SEQ ID NO: 1 - DNA, 22 bp, Artificial Sequence
  Synthetic Sequence
  misc_feature (7)..(7): 2′-deoxy-7-deazaguanosine
  misc_feature (11)..(12): Linked by a glycerol linker
  misc_feature (16)..(16): 2′-deoxy-7-deazaguanosine
tctgacgttcttcttgcagtct

SEQ ID NO: 2 - DNA, 22 bp, Artificial Sequence
  Synthetic Sequence
  misc_feature (7)..(7): 2′-deoxy-7-deazaguanosine
  misc_feature (11)..(12): Linked by a glycerol linker
  misc_feature (16)..(16): 2′-deoxy-7-deazaguanosine
tctgtcgttcttcttgctgtct

SEQ ID NO: 3 - DNA, 22 bp, Artificial Sequence
  Synthetic Sequence
  misc_feature (3)..(3): 2′-deoxy-7-deazaguanosine
  misc_feature (6)..(6): 2′-deoxy-7-deazaguanosine
  misc_feature (11)..(12): Linked by a glycerol linker
  misc_feature (17)..(17): 2′-deoxy-7-deazaguanosine
  misc_feature (20)..(20): 2′-deoxy-7-deazaguanosine
tcgtcgttctggtcttgctgct

SEQ ID NO: 4 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(12): Linked by is a glycerol linker
  misc_feature (11)..(11): 2′-deoxy-7-deazaguanosine
  misc_feature (12)..(12): 2′-deoxy-7-deazaguanosine
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 5 - DNA, 22 bp, Artificial Sequence
  Synthetic Sequence
  misc_feature (5)..(5): phosphodiester linkage
  misc_feature (6)..(6): 2′-deoxy-arabinoguanosine
  misc_feature (11)..(12): Linked by a glycerol linker
  misc_feature (17)..(17): 2′-deoxy-arabinoguanosine
  misc_feature (18)..(18): Phosphodiester linkage
ctgtcgttctcctcttgctgtc

SEQ ID NO: 6 - DNA, 22 bp, Artificial Sequence
  Synthetic Sequence
  misc_feature (6)..(6): 2′-deoxy-arabinoguanosine
  misc_feature (11)..(11): a phosphodiester linkage
  misc_feature (11)..(12): Linked by a glycerol linker
  misc_feature (12)..(12): Phosphodiester linkage
  misc_feature (17)..(17): 2′-deoxy-arabinoguanosine
ctgtcgttctcctcttgctgtc

SEQ ID NO: 7 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(12): Linked by a glycerol linker
  misc_feature (11)..(11): 2′-deoxy-7-deazaguanosine
  misc_feature (16)..(16): 2′-deoxy-arabinoguanosine
tcgaacgttcgtcttgctgtct

SEQ ID NO: 8 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a C3-linker
  misc_feature (16)..(16): 2′-deoxy-7-deazaguanosine
tcgaacgttcggacagctgtct

SEQ ID NO: 9 - DNA, 22 bp, Artificial Sequence
  Synthetic Sequence
  misc_feature (6)..(6): 2′-deoxy-arabinoguanosine
  misc_feature (11)..(12): Linked by a glycerol linker
  misc_feature (17)..(17): 2′-deoxy-arabinoguanosine
cagtcgttcaggacttgctgac

SEQ ID NO: 10 - DNA, 22 bp, Artificial Sequence
  Synthetic Sequence
  misc_feature (6)..(6): 2′-deoxy-7-deazaguanosine
  misc_feature (11)..(12): Linked by a glycerol linker
  misc_feature (17)..(17): 2′-deoxy-7-deazaguanosine
cagtcgttcaggacttgctgac

SEQ ID NO: 11 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (10)..(10): a phosphodiester linkage
  misc_feature (11)..(11): 2′-O-methylribonucleotide
  misc_feature (11)..(12): Linked by a 1,3,5-pentanetriol linker
  misc_feature (12)..(12): 2′-O-methylribonucleotide
  misc_feature (13)..(13): a phosphodiester linkage
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 12 - DNA, 26 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a 1,3-propanediol linker
  misc_feature (16)..(16): 2′-deoxy-7-deazaguanosine
  misc_feature (21)..(21): 2′-O-methylribonucleotide
  misc_feature (24)..(24): 2′-deoxy-7-deazaguanosine
tcgaacgttcgtcttgctgtcttgct

SEQ ID NO: 13 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a 1,3-propanediol linker
  misc_feature (16)..(16): 2′-deoxy-7-deazaguanosine
  misc_feature (20)..(20): 2′-O-methylribonucleotide
tcgaacgttcgtcttgctguct

SEQ ID NO: 14 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (8)..(8): a phosphodiester linkage
  misc_feature (10)..(10): a phosphodiester linkage
  misc_feature (11)..(11): 2′-O-methylribonucleotide
  misc_feature (11)..(12): Linked by a cis,trans-1,3,5-cyclohexanetriol linker
  misc_feature (12)..(12): 2′-O-methylribonucleotide
  misc_feature (13)..(13): a phosphodiester linkage
  misc_feature (14)..(14): a phosphodiester linkage
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 15 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (10)..(10): a phosphodiester linkage
  misc_feature (11)..(11): 2′-O-methylribonucleotide
  misc_feature (11)..(12): Linked by a 1,4-butanediol linker
  misc_feature (17)..(17): 2′-deoxy-arabinoguanosine
tcgaacgttcggacttgctgac

SEQ ID NO: 16 - DNA, 26 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (16)..(16): 2′-deoxy-7-deazaguanosine
  misc_feature (21)..(21): 2′-O-methylribonucleotide
  misc_feature (24)..(24): 2′-deoxy-7-deazaguanosine
tcgaacgttcgtgttgctgtcttgct

SEQ ID NO: 17 - DNA, 20 bp, Artificial Sequence
  Synthetic Sequence
  misc_feature (3)..(3): 2′-deoxy-arabinoguanosine
  misc_feature (6)..(6): 2′-deoxy-arabinoguanosine
  misc_feature (9)..(9): 2′-deoxy-U
  misc_feature (10)..(11): Linked by a cis,cis-1,3,5-cyclohexanetriol linker
  misc_feature (12)..(12): 2′-deoxy-U
  misc_feature (14)..(14): 2′-deoxy-arabinoguanosine
  misc_feature (17)..(17): 2′-deoxy-arabinoguanosine
tcgtcgttuyyuttgctgct

SEQ ID NO: 18 - DNA, 22 bp, Artificial Sequence
  Synthetic Sequence
  misc_feature (6)..(6): 2′-deoxy-arabinoguanosine
  misc_feature (11)..(12): Linked by a 1,4-butanediol linker
  misc_feature (16)..(16): 2′-deoxy-7-deazaguanosine
cagtcgttcagtcttgctgtct

SEQ ID NO: 19 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a glycerol linker
  misc_feature (12)..(12): 2′-deoxy-7-deazaguanosine
  misc_feature (16)..(16): 2′-deoxy-7-deazaguanosine
  misc_feature (20)..(20): 2′-deoxy-7-deazaguanosine
tcgtacgtacggcatgcatgct

SEQ ID NO: 20 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(12): Linked by a 1,3,5-pentanetriol linker
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 21 - DNA, 25 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (10)..(10): a phosphodiester linkage
  misc_feature (11)..(12): Linked by a 1,4-butanediol linker
  misc_feature (15)..(15): 2′-deoxy-arabinoguanosine
  misc_feature (23)..(23): 2′-deoxy-7-deazaguanosine
tcgaacgttcgcttgctgacttgct

SEQ ID NO: 22 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (7)..(7): a phosphodiester linkage
  misc_feature (11)..(11): 2′-deoxy-7-deazaguanosine
  misc_feature (11)..(12): Linked by a isobutanetriol linker
  misc_feature (12)..(12): 2′-deoxy-7-deazaguanosine
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 23 - DNA, 26 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a 1,5-pentandiol linker
  misc_feature (16)..(16): 2′-deoxy-7-deazaguanosine
  misc_feature (24)..(24): 2′-deoxy-7-deazaguanosine
tcgaacgttcgcattgctgtcttgct

SEQ ID NO: 24 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(11): 2′-deoxy-7-deazaguanosine
  misc_feature (11)..(12): Linked by a cis,trans-1,3,5-cyclohexanetriol linker
  misc_feature (12)..(12): 2′-deoxy-7-deazaguanosine
  misc_feature (16)..(16): 2′-deoxy-7-deazaguanosine
  misc_feature (20)..(20): 2′-deoxy-7-deazaguanosine
tcgaacgttcggcttgcaagct

SEQ ID NO: 25 - DNA, 22 bp, Artificial Sequence
  Synthetic Sequence
  misc_feature (2)..(2): a phosphodiester linkage
  misc_feature (3)..(3): a phosphodiester linkage
  misc_feature (3)..(3): 2′-deoxy-7-deazaguanosine
  misc_feature (6)..(6): a phosphodiester linkage
  misc_feature (7)..(7): 2′-deoxy-7-deazaguanosine
  misc_feature (10)..(10): a phosphodiester linkage
  misc_feature (11)..(11): a phosphodiester linkage
  misc_feature (11)..(11): 2′-deoxy-7-deazaguanosine
  misc_feature (11)..(12): Linked by a isobutanetriol linker
  misc_feature (12)..(12): a phosphodiester linkage
  misc_feature (12)..(12): 2′-deoxy-7-deazaguanosine
  misc_feature (13)..(13): a phosphodiester linkage
  misc_feature (16)..(16): 2′-deoxy-7-deazaguanosine
  misc_feature (17)..(17): a phosphodiester linkage
  misc_feature (20)..(20): a phosphodiester linkage
  misc_feature (20)..(20): 2′-deoxy-7-deazaguanosine
  misc_feature (21)..(21): a phosphodiester linkage
tcgaacgttcggcttgcaagct

SEQ ID NO: 26 - DNA, 22 bp, Artificial Sequence
  Synthetic Sequence
  misc_feature (1)..(1): a phosphodiester linkage
  misc_feature (3)..(3): a phosphodiester linkage
  misc_feature (3)..(3): 2′-deoxy-7-deazaguanosine
  misc_feature (6)..(6): a phosphodiester linkage
  misc_feature (7)..(7): 2′-deoxy-7-deazaguanosine
  misc_feature (8)..(8): a phosphodiester linkage
  misc_feature (9)..(9): a phosphodiester linkage
  misc_feature (9)..(9): 2′-deoxy-7-deazaguanosine
  misc_feature (9)..(10): Linked by an isobutanetriol linker
  misc_feature (10)..(10): a phosphodiester linkage
  misc_feature (10)..(10): 2′-deoxy-7-deazaguanosine
  misc_feature (11)..(11): a phosphodiester linkage
  misc_feature (15)..(15): a phosphodiester linkage
  misc_feature (18)..(18): a phosphodiester linkage
  misc_feature (18)..(18): 2′-deoxy-7-deazaguanosine
  misc_feature (20)..(20): a phosphodiester linkage
tcgaacgttcggcttgcaagct

SEQ ID NO: 27 - DNA, 22 bp, Artificial Sequence
  Synthetic Sequence
  misc_feature (2)..(2): a phosphodiester linkage
  misc_feature (3)..(3): a phosphodiester linkage
  misc_feature (3)..(3): 2′-deoxy-7-deazaguanosine
  misc_feature (6)..(6): a phosphodiester linkage
  misc_feature (7)..(7): 2′-deoxy-7-deazaguanosine
  misc_feature (10)..(10): a phosphodiester linkage
  misc_feature (11)..(11): a phosphodiester linkage
  misc_feature (11)..(11): 2′-deoxy-7-deazaguanosine
  misc_feature (11)..(12): Linked by a cis,trans-1,3,5-cyclohexanetriol linker
  misc_feature (12)..(12): a phosphodiester linkage
  misc_feature (12)..(12): 2′-deoxy-7-deazaguanosine
  misc_feature (13)..(13): a phosphodiester linkage
  misc_feature (16)..(16): 2′-deoxy-7-deazaguanosine
  misc_feature (17)..(17): a phosphodiester linkage
  misc_feature (20)..(20): a phosphodiester linkage
  misc_feature (20)..(20): 2′-deoxy-7-deazaguanosine
  misc_feature (21)..(21): a phosphodiester linkage
tcgaacgttcggcttgcaagct

SEQ ID NO: 28 - DNA, 22 bp, Artificial Sequence
  Synthetic Sequence
  misc_feature (2)..(2): a phosphodiester linkage
  misc_feature (3)..(3): a phosphodiester linkage
  misc_feature (3)..(3): 2′-deoxy-arabinoguanosine
  misc_feature (6)..(6): a phosphodiester linkage
  misc_feature (7)..(7): 2′-deoxy-arabinoguanosine
  misc_feature (10)..(10): a phosphodiester linkage
  misc_feature (11)..(11): a phosphodiester linkage
  misc_feature (11)..(11): 2′-deoxy-arabinoguanosine
  misc_feature (11)..(12): Linked by an isobutanetriol linker
  misc_feature (12)..(12): a phosphodiester linkage
  misc_feature (12)..(12): 2′-deoxy-arabinoguanosine
  misc_feature (13)..(13): a phosphodiester linkage
  misc_feature (16)..(16): 2′-deoxy-arabinoguanosine
  misc_feature (17)..(17): a phosphodiester linkage
  misc_feature (20)..(20): a phosphodiester linkage
  misc_feature (20)..(20): 2′-deoxy-arabinoguanosine
  misc_feature (21)..(21): a phosphodiester linkage
tcgaacgttcggcttgcaagct

SEQ ID NO: 29 - DNA, 22 bp, Artificial Sequence
  Synthetic Sequence
  misc_feature (2)..(2): a phosphodiester linkage
  misc_feature (3)..(3): a phosphodiester linkage
  misc_feature (3)..(3): 2′-deoxy-7-deazaguanosine
  misc_feature (6)..(6): a phosphodiester linkage
  misc_feature (7)..(7): 2′-deoxy-7-deazaguanosine
  misc_feature (10)..(10): a phosphodiester linkage
  misc_feature (11)..(11): a phosphodiester linkage
  misc_feature (11)..(12): Linked by a 1,3,5-pentanetriol linker
  misc_feature (12)..(12): a phosphodiester linkage
  misc_feature (13)..(13): a phosphodiester linkage
  misc_feature (16)..(16): 2′-deoxy-7-deazaguanosine
  misc_feature (17)..(17): a phosphodiester linkage
  misc_feature (20)..(20): a phosphodiester linkage
  misc_feature (20)..(20): 2′-deoxy-7-deazaguanosine
  misc_feature (21)..(21): a phosphodiester linkage
tcgaacgttcggcttgcaagct

SEQ ID NO: 30 - DNA, 22 bp, Artificial Sequence
  Synthetic Sequence
  misc_feature (1)..(1): a phosphodiester linkage
  misc_feature (3)..(3): a phosphodiester linkage
  misc_feature (3)..(3): 2′-deoxy-7-deazaguanosine
  misc_feature (6)..(6): a phosphodiester linkage
  misc_feature (7)..(7): 2′-deoxy-7-deazaguanosine
  misc_feature (10)..(10): a phosphodiester linkage
  misc_feature (11)..(11): a phosphodiester linkage
  misc_feature (11)..(12): Linked by a 1,3,5-pentanetriol linker
  misc_feature (12)..(12): a phosphodiester linkage
  misc_feature (13)..(13): a phosphodiester linkage
  misc_feature (16)..(16): 2′-deoxy-7-deazaguanosine
  misc_feature (17)..(17): a phosphodiester linkage
  misc_feature (20)..(20): a phosphodiester linkage
  misc_feature (20)..(20): 2′-deoxy-7-deazaguanosine
  misc_feature (21)..(21): a phosphodiester linkage
tcgaacgttcggcttgcaagct

(19) United States
(12) Patent Application Publication
(10) Pub. No.: US 2021/0340544 A1
(43) Pub. Date: Nov. 4, 2021

(54) TLR9 MODULATORS FOR TREATING CANCER

(71) Applicant: IDERA PHARMACEUTICALS, INC., Exton, PA (US)

(72) Inventors: Cara HAYMAKER, Pearland, TX (US); Chantale BERNATCHEZ, Pearland, TX (US); Adi DIAB, Houston, TX (US); Srinivas CHUNDURU, West Chester, PA (US)

(73) Assignee: IDERA PHARMACEUTICALS, INC., Exton, PA (US)

(21) Appl. No.: 17/286,209

(22) PCT Filed: Oct. 18, 2019
(86) PCT No.: PCT/US2019/057015

§ 371 Date: Apr. 16, 2021

Related U.S. Application Data
(60) Provisional application No. 62/747,627, filed on Oct. 18, 2018.
(60) Provisional application No. 62/775,792, filed on Dec. 5, 2018.

(51) Int. Cl. C12N 15/117; A61K 45/06; C07K 16/28

(52) U.S. Cl. CPC C12N 15/117; C07K 16/2818; A61K 45/06

(57) ABSTRACT

The present disclosure relates to methods for treating cancer in patients having low expression of MHC Class I genes, and in patients having increased serum levels of PD-L2 by administration of a TLR9 agonist.

### CROSS-REFERENCE TO RELATED APPLICATIONS

This application claims priority to U.S. Provisional Application No. 62/747,627, filed on Oct. 18, 2018, and U.S. Provisional Application No. 62/775,792, filed on Dec. 5, 2018, each of which is herein incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: 105968-5178 PR01_Sequence_Listing).

### FIELD OF THE INVENTION

The invention relates to the field of oncology, and use of immunotherapy in the treatment of cancer.

### BACKGROUND OF THE INVENTION

Toll-like receptors (TLRs) are present on many cells of the immune system and are involved in the innate immune response. In vertebrates, this family consists of eleven proteins called TLR1 to TLR11 that recognize pathogen associated molecular patterns from bacteria, fungi, parasites, and viruses. TLRs are a key mechanism by which vertebrates recognize and mount immune responses to foreign molecules and also provide a link between the innate and adaptive immune responses. Some TLRs are located on the cell surface to detect and initiate a response to extracellular pathogens and other TLRs are located inside the cell to detect and initiate a response to intracellular pathogens.

TLR9 recognizes unmethylated CpG motifs in bacterial DNA and in synthetic oligonucleotides. While agonists of TLR9, and other TLR agonists, can initiate anti-tumor immune responses, TLR agonists can also induce immune suppressive factors that may be counterproductive for effective tumor responses.

There is a need for cancer immunotherapies that induce antitumor responses, and keep the immune system productively engaged to improve the overall response. Additionally, there is a need to identify patients who may best benefit from such cancer immunotherapies and be more likely to respond to treatment.

### SUMMARY OF THE INVENTION

In various aspects, the present invention provides a method for treating a tumor, including, without limitation, metastatic melanoma, comprising intratumorally administering an oligonucleotide TLR9 agonist (e.g., IMO-2125 or other immunostimulatory oligonucleotides described herein) to a cancer patient. The method further comprises administering an immune checkpoint inhibitor therapy, such as a therapy targeting CTLA-4, PD-1/PD-L1/PD-L2, TIM3, LAG3, and/or IDO. The TLR9 agonist upon intratumoral injection induces global increases in expression of checkpoint genes, including IDO1, PDL1, PD1, IDO2, CEACAM1, OX40, TIM3, LAG3, CTLA4, and OX40L. By altering immune signaling in the tumor microenvironment, such changes in gene expression provide opportunities to improve responsiveness to checkpoint inhibitor therapy, including in some embodiments, a complete response. The invention further provides the opportunity to balance anti-tumor responses with inhibitory signals, thereby also minimizing immune-related adverse events (irAEs) of checkpoint inhibitor therapy. The invention further provides the opportunity to select patients with metastatic disease whose tumor is more likely to respond to therapy.

In various embodiments, the patient has a cancer that was previously unresponsive to, or had become resistant to, a checkpoint inhibitor therapy, such as anti-CTLA-4, anti-PD-1, or anti-PD-L1 and/or anti-PD-L2 agent. The invention finds use for treating primary cancer or a metastatic cancer, including cancers that originate from skin, colon, breast, or prostate, among other tissues. In some embodiments, the cancer is progressive, locally advanced, or metastatic carcinoma. In some embodiments, the cancer is metastatic melanoma.

In accordance with embodiments of the invention, the immunostimulatory oligonucleotide (e.g., IMO-2125) is administered intratumorally. Intratumoral administration alters immune signaling in the tumor microenvironment, priming the immune system for an effective anti-tumor response, while inducing changes that are compatible with more effective checkpoint inhibitor therapy. For example, the TLR9 agonist (e.g., IMO-2125) may be administered intratumorally at from about 4 mg to about 64 mg per dose, with from about 3 to about 12 doses being administered over 10 to 12 weeks. For example, therapy may be initiated with 3 to 5 weekly doses of IMO-2125, optionally followed by 3 to 8 maintenance doses, which are administered about every three weeks.

During the regimen of IMO-2125 (or other TLR9 agonist), one or more checkpoint inhibitor therapies are administered to take advantage of the changes in immune signaling. In some embodiments, the patient receives an anti-CTLA-4 agent (e.g., ipilimumab or tremelimumab) and/or an anti-PD-1 agent (e.g., nivolumab or pembrolizumab). The immune checkpoint inhibitor can be administered parenterally, such as, in some embodiments, subcutaneously, intratumorally, intravenously. For example, in various embodiments the immune checkpoint inhibitor is administered at a dose of from about 1 mg/kg to about 5 mg/kg intravenously. The initial dose of the immune checkpoint inhibitor can be administered at least one week after the initial TLR9 agonist dose, for example in about weeks 2, 3 or 4. In some embodiments, the immunotherapy agent is administered from about 2 to about 6 times (e.g., about 4 times, preferably every three weeks).

In some embodiments, IMO-2125 is administered intratumorally to a metastatic melanoma patient previously found to be unresponsive or only partially responsive to PD-1 blockade therapy. For example, IMO-2125 is administered at a dose of from 4 to 32 mg per dose in weeks 1, 2, 3, 5, 8, and 11, with ipilimumab i.v. at 3 mg/kg. Ipilimumab can be administered every three weeks, beginning in week 2. Alternatively, pembrolizumab can be administered i.v. at 2 mg/kg every three weeks beginning on week 2.

In some embodiments, IMO-2125 is administered intratumorally to a metastatic melanoma patient exhibiting low expression of MHC Class I genes, e.g., in a tumor biopsy. For example, IMO-2125 is administered at a dose of from 4 to 32 mg per dose in weeks 1, 2, 3, 5, 8, and 11, with ipilimumab i.v. at 3 mg/kg. Ipilimumab can be administered every three weeks, beginning in week 2. Alternatively, pembrolizumab can be administered i.v. at 2 mg/kg every three weeks beginning on week 2.

In some embodiments, IMO-2125 is administered intratumorally to a metastatic melanoma patient exhibiting no measurable expression of HLA-A, HLA-B, and HLA-C, e.g., in a tumor biopsy. In some embodiments, IMO-2125 is administered intratumorally to a metastatic melanoma patient exhibiting no measurable expression of B2M, the β2-microglobulin gene, e.g., in a tumor biopsy. In another aspect, IMO-2125 is administered to metastatic cancer patients exhibiting elevated levels of serum PD-L2. In another aspect, IMO-2125 is administered to metastatic cancer patients with tumors enriched for dendritic cells as determined by pre-treatment biopsy analysis.

The present methods in various embodiments allow for a robust anti-tumor immune response (which in some embodiments is a complete response), and which does not come at the expense of significant side effects, e.g. relative to side effects observed when one or more immunotherapies are used in the absence of the TLR9 agonist. Such side effects include commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease (among others).

In an embodiment of the invention, a method for treating a tumor in a patient having low tumor expression of MHC Class I genes comprising intratumoral administration of a TLR 9 agonist is disclosed. In some embodiments, a method for treating a tumor in a patient comprising: (a) determining MHC Class I gene expression in a tumor sample and (b) administering a TLR9 agonist if said gene expression is present in less than 50% of the tumor cells is disclosed.

In some embodiments according to the present invention, a method for treating a tumor in a patient having increased serum PD-L2 levels comprising intratumoral administration of a TLR 9 agonist is disclosed. In some embodiments according to the present invention, a method for treating a tumor in a patient having increased serum PD-L2 levels comprising: (a) determining serum PD-L2 levels in said patient and (b) administering a TLR 9 agonist if said PD-L2 levels are increased in said patient as compared with a control level. In some embodiments, the PD-L2 level is between about 750 pg/mL and 5000 pg/mL. In some embodiments, the PD-L2 level is between about 1100 pg/mL and about 3000 pg/mL. In some embodiments, the PD-L2 level is between about 1100 pg/mL and 2100 pg/mL. In some embodiments, the patient is selected based on a baseline tumor biopsy enriched in dendritic cells.

In any of the methods disclosed herein, the TLR9 agonist has the structure: 5′-TCG₁AACG₁TTCG₁-X-G₁CTTG₁CAAG₁CT-5′ (5′ SEQ ID NO:4-X-SEQ ID NO:4 5′), wherein G₁ is 2′-deoxy-7-deazaguanosine and X is a glycerol linker. In some embodiments, the TLR9 agonist is tilsotolimod (IMO-2125). In another embodiment, any of the methods disclosed herein further comprise administering at least one immune checkpoint inhibitor. In another embodiment, any of the methods disclosed herein further comprise first sensitizing the tumor microenvironment with intratumoral administration of the TLR9 agonist.

In some embodiments according to the present invention, the immune checkpoint inhibitor is co-administered with the TLR9 agonist. In some embodiments, immune checkpoint inhibitor is administered after the TLR9 agonist. In some embodiments, the immune checkpoint inhibitor is administered at least one day after the TLR9 agonist. In some embodiments, the immune checkpoint inhibitor is administered at least one week after the TLR9 agonist.

In some embodiments according to the present invention, the immune checkpoint inhibitor is selected from a checkpoint inhibitor that targets PD-1, PD-L1, cytotoxic T-lymphocyte-associated protein 4 (CTLA-4), LAG3, B7-H3, B7-H4, KIR, OX40, IgG, IDO-1, IDO-2, CEACAM1, TNFRSF4, BTLA, OX40L, and TIM3. In some embodiments, the checkpoint inhibitor targets CTLA-4 and is a monoclonal antibody against CTLA-4. In some embodiments, the checkpoint inhibitor is selected from the group consisting of ipilimumab, tremelimumab, or biosimilars thereof. In some embodiments, the checkpoint inhibitor targets PD-1 and is selected from the group consisting of nivolumab, pembrolizumab, and biosimilars thereof.

In some embodiments according to the present invention, the checkpoint inhibitor is administered beginning on week 2 after a first administration of TLR9 agonist. In some embodiments, the checkpoint inhibitor is administered beginning on week 3 after a first administration of TLR9 agonist. In some embodiments, the checkpoint inhibitor is administered every three weeks. In some embodiments, the checkpoint inhibitor is administered at least 2 to 6 times.

In some embodiments according to the present invention, the TLR9 agonist is administered at a dose of from about 1 mg to about 20 mg. In some embodiments, the dose is about 8 mg.

In some embodiments, the TLR 9 agonist is IMO-2125 and the immune checkpoint inhibitor therapy is an anti-CTLA4 inhibitor.

In some embodiments according to the present invention, the tumor is a metastatic tumor. In some embodiments, the tumor is selected from melanoma, lung tumor, kidney tumor, prostate tumor, cervical tumor, colorectal tumor, colon tumor, pancreatic tumor, ovarian tumor, urothelial tumor, gastric/GEJ tumor, head and neck tumor, glioblastoma, Merkel cell tumor, head and neck squamous cell carcinoma (HNSCC), non-small cell lung carcinoma (NSCLC), small cell lung tumor (SCLC), or bladder tumor. In some embodiments, the tumor is metastatic melanoma. In some embodiments, the tumor is a colorectal tumor or a colon tumor. In some embodiments, the tumor is a head and neck tumor or a head and neck squamous cell carcinoma (HNSCC).

In some embodiments according to the present invention, the low expression of tumor MHC Class I gene expression is less than 25% of the expression in healthy tissue. In some embodiments, the low expression of tumor MHC Class I gene expression is less than 50% of the expression in healthy tissue.

In some embodiment according to the present invention, a method for treating metastatic melanoma in a patient having 50% or lower tumor expression of MHC Class I genes, the method comprising: (a) sensitizing the tumor microenvironment with intratumoral administration of tilsotolimod (IMO-2125) at a dose of about 8 mg and (b) systemically administering ipilimumab at least one week after the administration of tilsotolimod is disclosed.

Other aspects and embodiments will be apparent from the following detailed description.

### BRIEF DESCRIPTION OF THE DRAWINGS

FIG. 1 shows IRF7 gene expression levels before an intratumoral dose of tilsotolimod and 24 hours after an intratumoral dose.

FIG. 2 shows a volcano plot of genes upregulated following an intratumoral dose of tilsotolimod. Among the upregulated genes are IRF7, MX1, IFIT1, IFIT2, TAP1, and TAP2.

FIG. 3 shows the Dendritic Cell (DC) score (log₂) of the baseline tumor lesions for patients showing either a complete response (CR) or a partial response (PR), those with progressive disease (PD), and those with stable disease (SD) after treatment.

FIG. 4 shows the results of flow cytometry analysis of tumor biopsy tissues both before an intratumoral dose of tilsotolimod and 24 hours after an intratumoral dose. The percentage of cells expressing HLA-DR are reported.

FIGS. 5A-5C show HLA-A, HLA-B, and HLA-C gene expression, respectively, in tumors from patients showing either a complete response (CR) or a partial response (PR), those with progressive disease (PD), and those with stable disease (SD) after treatment.

FIG. 6 shows a heatmap of the cytotoxicity gene expression profile in baseline tumor samples. The heatmap is shaded based on clinical response.

FIG. 7 comprises FIGS. 7A-7E. FIG. 7A shows imaging guided intratumoral injection of IMO-2125. FIG. 7B shows two pre- and post-therapy injected (yellow arrow) and distant (red arrow) lesions. FIG. 7C depicts the RECIST v1.1 classification of the decrease in target lesion diameters for the study participants in change from baseline (percentage, %). FIG. 7D plots the best response as of the cut-off date for study subjects with at least one post-baseline disease evaluation. FIG. 7E is a waterfall plot showing the maximum percentage reduction from baseline sum of the individual longest lesion diameters (mm) by injection status, where, at each point, the bars on the left represent injected measurable lesions and the bars on the right represent non-injected measurable lesions.

FIG. 8 comprises FIGS. 8A-8E, and demonstrates IMO-2125 induces a local type 1 IFN response gene signature, macrophage influx and DC1 maturation. FIG. 8A shows a schematic of the tissue and blood samples collected from subjects during the course of the study. Light arrows depict tumor biopsy collection and dark arrows show collection of peripheral blood mononuclear cells (PBMCs). FIG. 8B is a volcano plot of RNA extracted from the local injected lesion at 24 h post IMO-2125 as compared to the same lesion at baseline (predose). The adjusted p-value is shown. FIG. 8C shows the macrophage score as determined using the nSolver advanced analysis tool and is shown on a log₂ scale. FIG. 8D shows the predose (baseline) and 24-hour postsdose percentage HDR-DR expressed on live, lineage neg, CD1c+mDC1 cells. A minimum of 100 events was required for subgating. Finally, FIG. 8E shows the number of cells/mm² expressing IDO as assessed by a chromogenic immunohistochemistry (IHC) assay.

FIG. 9 comprises FIGS. 9A-9E and illustrate that local DC presence at baseline and combination therapy overcomes known mechanisms of resistance to single agent anti-CTLA4. FIG. 9A plots the DC (dendritic cell) score determined using the nSolver advanced analysis tool and shown on a log₂ scale. FIG. 9B plots the concentration of soluble PD-L2 in patient plasma measured before treatment. FIG. 9C plots the cell type score of each major cell type at baseline in local lesions as determined by the nSolver advanced analysis tool and is plotted based on patient clinical response. Complete Response (CR)+Partial Response (PR); Progressed Disease (PD); and Stable Disease (SD). FIG. 9D is a heatmap produced by hierarchical clustering using the T-cell functionality gene set at baseline in both local and distant lesions. FIG. 9E is a second hierarchal clustering heatmap based on the cytotoxicity gene set at baseline in both local and distant lesions.

FIG. 10 comprises FIGS. 10A-10D and illustrate data from banked PBMCs collected prior to-and-during treatment. The PBMCs were thawed and stained for memory/differentiation status and sorted using flow cytometry. The horizontal line in each of FIGS. 10B-10D indicates the median frequency across all the patients at a given time point. Each patient is indicated by their study ID. FIG. 10A is a representative dot plot showing memory subset identification by co-expression patterns of CCR7 and CD45RA with the live, CD45+CD3+CD8+ subset. FIGS. 10B through 10D show the frequency of the T_{EM} subset over time in responding patients (PR+CR) (FIG. 10B), SD patients (FIG. 10C), and PD patients (FIG. 10D).

FIG. 11 comprises FIGS. 11A-11D and illustrates that tumor infiltrating lymphocyte (TIL) activation and proliferation correlates with response to combination therapy. Unsupervised hierarchical clustering based on Nanostring gene expression profiling: FIG. 11A shows a T-cell functional gene signature; and FIG. 11B shows a cytotoxicity gene signature. FIGS. 11C and 11D depict proliferation as measured using Ki67 staining and sorting by flow cytometry of CD8+ TILs at baseline, 24 h after intratumoral injection and at C3W8 in either tumor lesions (p=0.0071) or in PBMCs (p>0.05) at baseline and C3W8, for responders (FIG. 11C) and non-responders (FIG. 11D).

FIG. 12 comprises FIGS. 12A-12C. FIGS. 12A and 12B show the frequency of the top 50 clones in the distant tumor lesions at C3W8 compared to their initial frequency at baseline for responding (FIG. 12A) and non-responding (FIG. 12B) patients. FIG. 12C illustrates individual T-cell clones (top 50) identified at C3W8 in the distant lesions of individual responding patients assessed for presence in the local/injected lesion (baseline and C3W8) and at baseline in the distant lesion. Each image represents individual patients with each circle representing an individual T cell clone. Clones shared between lesions at all time points are shown in blue. The size of the circle indicates relative frequency at C3W8 and the numbers indicate the frequency relative to initial baseline presence.

FIG. 13 Compares PD-L1 staining prior to therapy (at baseline) to that 24 h post injection in the injected lesion. Chromogenic IHC staining PD-L1 of injected tumor lesions prior to therapy and 24 h post IMO-2125 injection. PD-L1 is indicated as a percentage of the tumor cells present as indicated by H&E.

FIG. 14 comprises FIGS. 14A and 14B and shows Immunohistochemistry (IHC) staining of CD3+ and CD8+ of injected and distant tumor lesions prior to therapy. The closed circles indicate stable disease (SD) and progressive disease (PD) patients. Open circles indicate partial response (PR) and compete response (CR) patients. Each point represents the mean of the total areas assessed in cells/mm². FIG. 14A shows CD3 staining and FIG. 14B shows CD8 staining. Only samples with tumor presence as indicated by H&E are shown.

FIG. 15 comprises FIGS. 15A-15C and illustrates normalized, linear reads of the baseline tumor lesions (both local/injected and distant): FIG. 15A shows HLA-A expression; FIG. 15B shows HLA-B expression; and FIG. 15C HLA-C expression stratified based upon subsequent confirmed clinical response.

FIG. 16 comprises FIGS. 16A and 16B. FIG. 16A is a heatmap of the global pathways assessed. The induction of a macrophage function score at cycle 3 week 8 as compared to baseline tumor tissue is shown in FIG. 16B. Each spot represents a single patient sample and is in a log₂ scale.

### DETAILED DESCRIPTION

### Definitions

The term “3′”, when used directionally, generally refers to a region or position in a polynucleotide or oligonucleotide 3′ (toward the 3′ position of the oligonucleotide) from another region or position in the same polynucleotide or oligonucleotide.

The term “5′”, when used directionally, generally refers to a region or position in a polynucleotide or oligonucleotide 5′ (toward the 5′ position of the oligonucleotide) from another region or position in the same polynucleotide or oligonucleotide.

The term “about” generally means plus or minus 10% of an associated numerical value or numerical value range.

The term “agonist” generally refers to a substance that binds to a receptor of a cell and induces a response. Such response may be an increase in the activity mediated by the receptor. An agonist often mimics the action of a naturally occurring substance such as a ligand.

The term “antagonist” or “inhibitor” generally refers to a substance that can bind to a receptor, but does not produce a biological response upon binding. The antagonist or inhibitor can block, inhibit, or attenuate the response mediated by an agonist and may compete with agonist for binding to a receptor. Such antagonist or inhibitory activity may be reversible or irreversible.

The term “antigen” generally refers to a substance that is recognized and selectively bound by an antibody or by a T cell antigen receptor. Antigens may include but are not limited to peptides, proteins, nucleosides, nucleotides and combinations thereof. Antigens may be natural or synthetic and generally induce an immune response that is specific for that antigen.

The term “cancer” generally refers to, without limitation, any malignant growth or tumor caused by abnormal or uncontrolled cell proliferation and/or division. Cancers may occur in humans and/or animals and may arise in any and all tissues. Treating a patient having cancer with the invention may include administration of a compound, pharmaceutical formulation or vaccine according to the invention such that the abnormal or uncontrolled cell proliferation and/or division is affected.

The term an “effective amount” generally refers to an amount sufficient to affect a desired biological effect, such as a beneficial result. Thus, an “effective amount” will depend upon the context in which it is being administered. A effective amount may be administered in one or more prophylactic or therapeutic administrations.

The term “in combination with” generally means administering a first agent and another agent useful for treating the disease or condition.

The term “individual”, “patient”, or “subject” are used interchangeably and generally refers to a mammal, such as a human. Mammals generally include, but are not limited to, humans, non-human primates, rats, mice, cats, dogs, horses, cattle, cows, pigs, sheep and rabbits.

The term “linker” generally refers to any moiety that can be attached to an oligonucleotide by way of covalent or non-covalent bonding through a sugar, a base, or the backbone. The linker can be used to attach two or more nucleosides or can be attached to the 5′ and/or 3′ terminal nucleotide in the oligonucleotide. In certain embodiments of the invention, such linker may be a non-nucleotidic linker.

The term “non-nucleotidic linker” generally refers to a chemical moiety other than a nucleotidic linkage that can be attached to an oligonucleotide by way of covalent or non-covalent bonding. Preferably such non-nucleotidic linker is from about 2 angstroms to about 200 angstroms in length, and may be either in a cis or trans orientation.

The term “nucleotidic linkage” generally refers to a chemical linkage to join two nucleosides through their sugars (e.g. 3′-3′, 2′-3′,2′-5′, 3′-5′) consisting of a phosphorous atom and a charged, or neutral group (e.g., phosphodiester, phosphorothioate or phosphorodithioate) between adjacent nucleosides.

The term “treatment” generally refers to an approach intended to obtain a beneficial or desired result, which may include alleviation of symptoms, or delaying or ameliorating a disease progression.

As used herein, the term “TLR9 agonist” generally refers to an immunostimulatory oligonucleotide compound comprising a CpG dinucleotide motif and is able to enhance or induce an immune stimulation mediated by TLR9. In some embodiments the CpG dinucleotide is selected from the group consisting of CpG, C*pG, CpG*, and C*pG*, wherein C is 2′-deoxycytidine, C* is an analog thereof, G is 2′-deoxyguanosine, and G* is an analog thereof, and p is an internucleoside linkage selected from the group consisting of phosphodiester, phosphorothioate, and phosphorodithioate. In preferred embodiments C* is selected from the group consisting of 2′-deoxythymidine, arabinocytidine, 2′-deoxythymidine,2′-deoxy-2′-substituted arabinocytidine, 2′-O-substituted arabinocytidine,2′-deoxy-5-hydroxycytidine, 2′-deoxy-N4-alkyl-cytidine,2′-deoxy-4-thiouridine. In preferred embodiments, G* is 2′deoxy-7-deazaguanosine, 2′-deoxy-6-thioguanosine, arabinoguanosine,2′-deoxy-2′substituted-arabinoguanosine,2′-O-substituted-arabinoguanosine, 2′-deoxyinosine. In certain preferred embodiments, the immunostimulatory dinucleotide is selected from the group consisting of C*pG, CpG*, and C*pG*.

As used herein, an immunomer refers to a compound comprising at least two oligonucleotides linked together through their 3′ ends, such that the immunomer has more than one accessible 5′ end, wherein at least one of the oligonucleotides is an immunostimulatory oligonucleotide. The linkage at the 3′ ends of the component oligonucleotides is independent of the other oligonucleotide linkages and may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 3′ terminal nucleotide. The term “accessible 5′ end” means that the 5′ end of the oligonucleotide is sufficiently available such that the factors that recognize and bind to immunomers and stimulate the immune system have access to it. Optionally, the 5′ OH can be linked to a phosphate, phosphorothioate, or phosphorodithioate moiety, an aromatic or aliphatic linker, cholesterol, or another entity which does not interfere with accessibility.

As used herein, an immunostimulatory oligonucleotide is an oligodeoxyribonucleotide that comprises a CpG dinucleotide motif and is capable of enhancing or inducing a TLR9-mediated immune response. In some embodiments the CpG dinucleotide is selected from the group consisting of CpG, C*pG, CpG*, and C*pG*, wherein C is 2′-deoxycytidine, C* is an analog thereof, G is 2′-deoxyguanosine, and G* is an analog thereof, and p is an internucleoside linkage selected from the group consisting of phosphodiester, phosphorothioate, and phosphorodithioate. In preferred embodiments C* is selected from the group consisting of 2′-deoxythymidine, arabinocytidine, 2′-deoxythymidine,2′-deoxy-2′-substitutedarabinocytidine, 2′-O-substitutedarabinocytidine,2′-deoxy-5-hydroxycytidine, 2′-deoxy-N4-alkyl-cytidine,2′-deoxy-4-thiouridine. In preferred embodiments, G* is 2′deoxy-7-deazaguanosine, 2′-deoxy-6-thioguanosine, arabinoguanosine,2′-deoxy-2′substituted-arabinoguanosine,2′-O-substituted-arabinoguanosine, 2′-deoxyinosine. In certain preferred embodiments, the immunostimulatory dinucleotide is selected from the group consisting of C*pG, CpG*, and C*pG*.

In some embodiments, the immunomer comprises two or more immunostimulatory oligonucleotides which may be the same or different. Preferably, each such immunostimulatory oligonucleotide has at least one accessible 5′ end.

In some embodiments, the oligonucleotides of the immunomer each independently have from about 3 to about 35 nucleoside residues, preferably from about 4 to about 30 nucleoside residues, more preferably from about 4 to about 20 nucleoside residues. In some embodiments, the oligonucleotides have from about 5 to about 18, or from about 5 to about 14, nucleoside residues. As used herein, the term “about” implies that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above. In some embodiments, one or more of the oligonucleotides have 11 nucleotides.

In certain embodiments of the invention, the immunomers comprise two oligonucleotides covalently linked by a nucleotide linkage, or anon-nucleotide linker, at their 3′-ends or by functionalized sugar or by functionalized nucleobase via a non-nucleotide linker or a nucleotide linkage. As a non-limiting example, the linker may be attached to the 3′-hydroxyl. In such embodiments, the linker comprises a functional group, which is attached to the 3′-hydroxyl by means of a phosphate-based linkage like, for example, phosphodiester, phosphorothioate, phosphorodithioate, methylphosphonate, or by non-phosphate-based linkages. Possible sites of conjugation for the ribonucleotide are indicated in Formula I, below, wherein B represents a heterocyclic base and wherein the arrow pointing to P indicates any attachment to phosphorous.

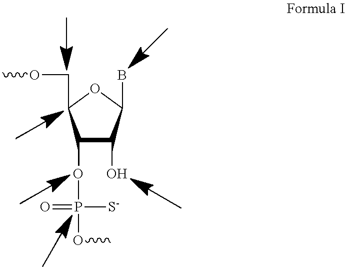

In some embodiments, the non-nucleotide linker is a small molecule, macromolecule or biomolecule, including, without limitation, polypeptides, antibodies, lipids, antigens, allergens, and oligosaccharides. In some other embodiments, the non-nucleotidic linker is a small molecule. For purposes of the invention, a small molecule is an organic moiety having a molecular weight of less than 1,000 Da. In some embodiments, the small molecule has a molecular weight of less than 750 Da.

In some embodiments, the small molecule is an aliphatic or aromatic hydrocarbon, either of which optionally can include, either in the linear chain connecting the oligoribonucleotides or appended to it, one or more functional groups including, but not limited to, hydroxy, amino, thiol, thioether, ether, amide, thioamide, ester, urea, or thiourea. The small molecule can be cyclic or acyclic. Examples of small molecule linkers include, but are not limited to, amino acids, carbohydrates, cyclodextrins, adamantane, cholesterol, haptens and antibiotics. However, for purposes of describing the non-nucleotidic linker, the term “small molecule” is not intended to include a nucleoside.

In some embodiments, the non-nucleotidic linker is an alkyl linker or amino linker. The alkyl linker may be branched or unbranched, cyclic or acyclic, substituted or unsubstituted, saturated or unsaturated, chiral, achiral or racemic mixture. The alkyl linkers can have from about 2 to about 18 carbon atoms. In some embodiments such alkyl linkers have from about 3 to about 9 carbon atoms. Some alkyl linkers include one or more functional groups including, but not limited to, hydroxy, amino, thiol, thioether, ether, amide, thioamide, ester, urea, and thioether. Such alkyl linkers can include, but are not limited to, 1,2 propanediol, 1,2,3 propanetriol, 1,3 propanediol, triethylene glycol hexaethylene glycol, polyethylene glycol linkers (e.g. [—O—CH2-CH2-]ₙ (n=1-9)), methyl linkers, ethyl linkers, propyl linkers, butyl linkers, or hexyl linkers. In some embodiments, such alkyl linkers may include peptides or amino acids.

In various aspects, the present invention provides a method for treating a tumor, e.g. a metastatic tumor (including, without limitation, metastatic melanoma) comprising intratumorally administering an oligonucleotide TLR9 agonist (e.g., IMO-2125) to a cancer patient, in combination with immunotherapy with an immune checkpoint inhibitor therapy, such as a therapy targeting CTLA-4, PD-1/PD-L1/PD-L2, LAG3, TIM3, and/or IDO, wherein the tumor has low MHC Class I expression.

In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS(phosphatidylserine), OX-40, SLAM, TIGHT, VISTA, VTCN1, or any combinations thereof. In some embodiments, the immune checkpoint inhibitor is an inhibitor of IDO1, CTLA4, PD-1, LAG3, PD-L1, TIM3, or combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of IDO1. In some embodiments, the one or more checkpoint inhibitors are administered by any suitable route. In some embodiments, the route of administration of the one or more checkpoint inhibitors is parenteral, mucosal delivery, oral, sublingual, transdermal, topical, inhalation, intranasal, aerosol, intratumoral, intraocular, intratracheal, intrarectal, intragastric, vaginal, by gene gun, dermal patch or in eye drop or mouthwash form. In some embodiments, the one or more TLR9 agonists and the one or more checkpoint inhibitors are each administered in a pharmaceutically effective amount.

Exemplary immune checkpoint inhibitors include anti-PD-1, anti-PD-L1, anti-PD-L2, and anti-CTLA-4 agents. PD-1/PD-L1/PD-L2 antibodies inhibit the interaction between PD-1 and its ligands (PD-L1 and PD-L2) on tumor cells to promote immune-mediated tumor destruction. CTLA-4 antibodies block the inhibitory signals to T-cells transmitted by CTLA-4. While PD-1 antibodies and CTLA-4 antibodies have emerged as important therapeutic options for a variety of cancers, many patients fail to respond. For example, some melanoma patients show no response to anti-PD-1 treatment, or even progress, after 12 weeks of treatment. Further, immune checkpoint blockade is associated with various immune-related adverse events, which can affect various tissues and organs including the skin, the gastrointestinal tract, the kidneys, peripheral and central nervous system, liver, lymph nodes, eyes, pancreas, and the endocrine system. These immune-related adverse events (irAEs) can be severe, or even fatal, and may require discontinuation of therapy. Examples of common irAEs are hypophysitis, colitis, hepatitis, pneumonitis, rash, and rheumatic disease.

Expression of the various immune checkpoint molecules on cells of the immune system induces a complex series of events that determines whether an immune response will be effective to combat the tumor, or otherwise result in immune tolerance. For example, increased expression of PD-1 on dendritic cells (DCs) promotes apoptosis of activated DCs, a critical antigen presenting cell for anti-tumor immune responses. Park S J, Negative role of inducible PD-1 on survival of activated dendritic cells, J. Leukocyte Biology 95(4):621-629 (2014). Further, expression of IDO, PD-L1, and CTLA-4 in the peripheral blood of melanoma patients and can be associated with advanced disease and negative outcomes, and are interconnected, suggesting that multiple immune checkpoints might require targeting to improve therapy in some cases. Chevolet I, et al., Characterization of the in vivo immune networks of IDO, tryptophan metabolism, PD-L1, and CTLA-4 in circulating immune cells in melanoma, Oncoimmunology 4(3) e982382-7 (2015).

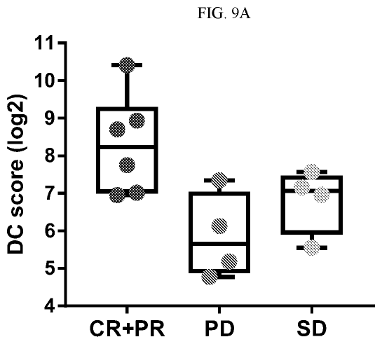

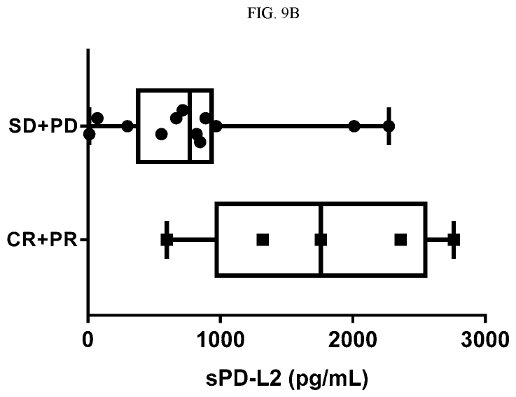

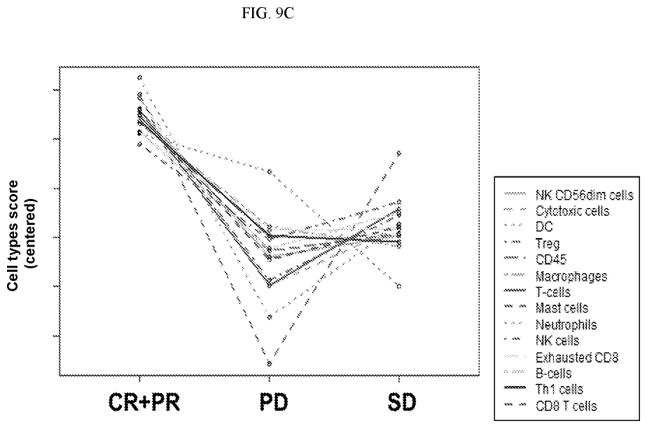

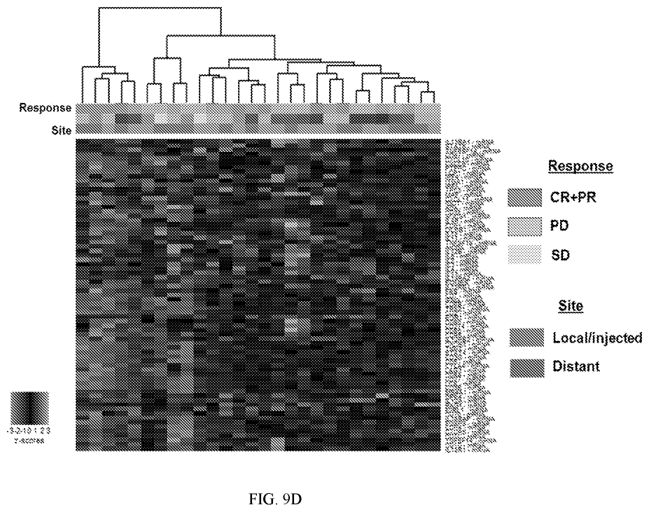

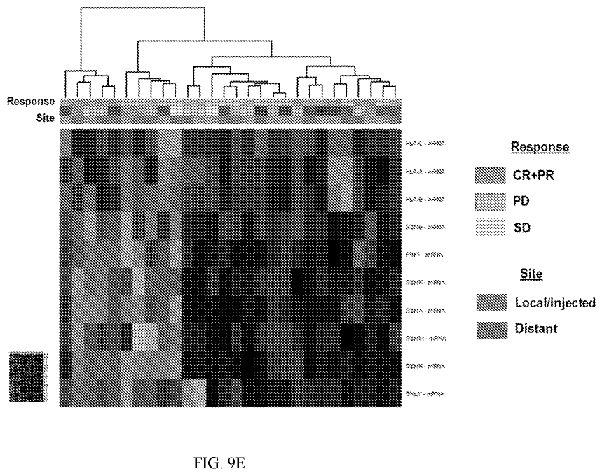

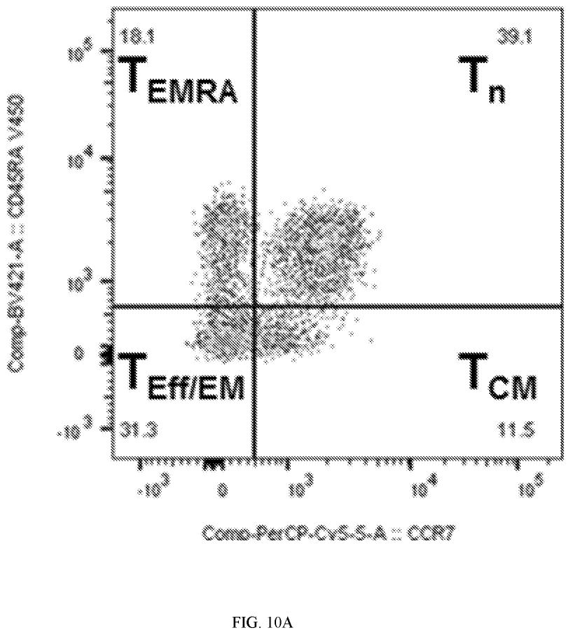

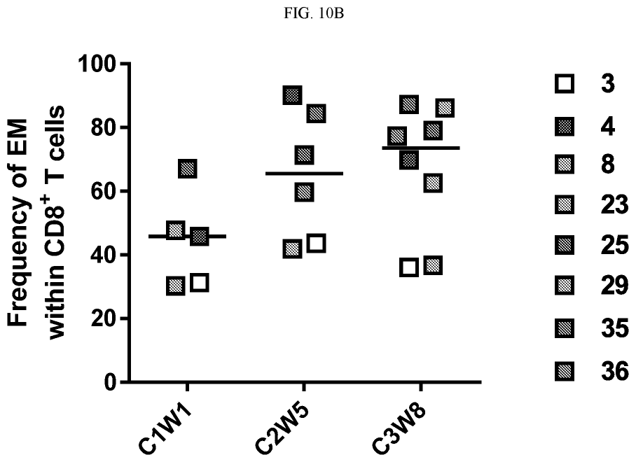

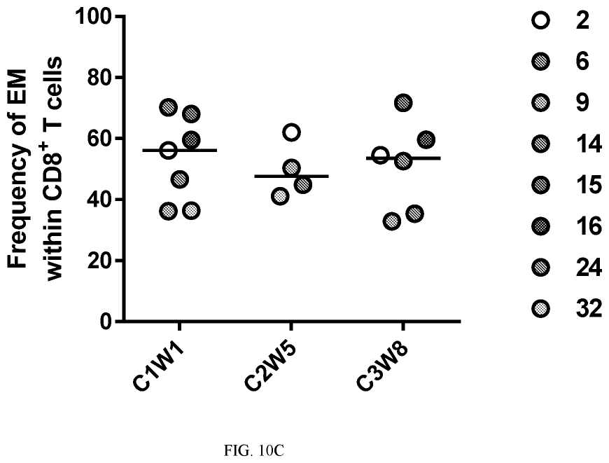

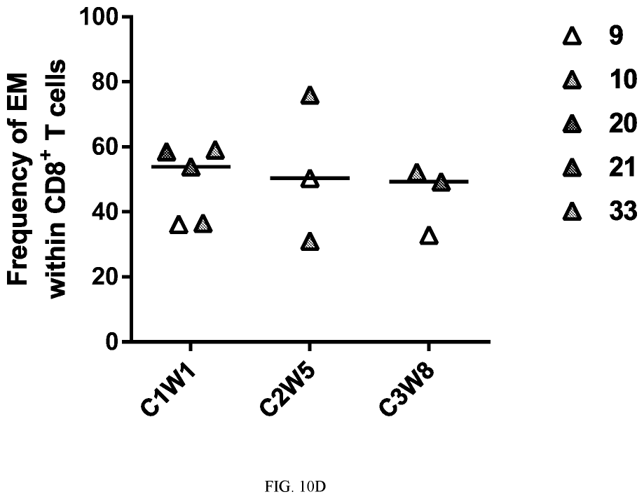

In some embodiments, the metastatic tumor has a high proportion of dendritic cells (DC) at baseline. In some embodiments, the metastatic tumor is enriched for dendritic cells before treatment with tilsotolimod (IMO-2125). Enrichment for dendritic cells in baseline metastatic tumors may be determined, for example, by analyzing a biopsy specimen with immunohistochemistry (IHC) or by disaggregating fresh biopsy specimens and using flow cytometry sorting cells bearing DC markers, for example, CD209, CCL13, HSD11B1, and CD11c⁺. FIG. 9A shows the level of dendritic cells in baseline tumor biopsy specimens. Metastatic tumors that responded to intratumoral IMO-2125 treatment in combination with systemic anti-CTLA4 treatment were enriched at baseline for dendritic cells in the tumor biopsy. In another aspect, metastatic melanoma patients who have progressive disease following treatment with one or more checkpoint inhibitors are selected for intratumoral IMO-2125 treatment based on dendritic cell enrichment in one or more progressive disease tumors.

In another aspect, the patient with metastatic cancer has elevated levels of PD-L2 protein in serum. In some embodiments, the elevated level of PD-L2 protein between about 750 pg/mL and about 5000 pg/mL. In some embodiments, the elevated level of PD-L2 protein above about 1000 pg/mL. In some embodiments, the elevated level is above about 1100 pg/mL, above about 1200 pg/mL, above about 1300 pg/mL, above about 1400 pg/mL, above about 1500 pg/mL, above about 1600 pg/mL, above about 1700 pg/mL, above about 1800 pg/mL, above about 1900 pg/mL, above about 2000 pg/mL, above about 2100 pg/mL, above about 2200 pg/mL, above about 2300 pg/mL, above about 2400 pg/mL, above about 2500 pg/mL, above about 2600 pg/mL, above about 2700 pg/mL, above about 2800 pg/mL, above about 2900 pg/mL, and above about 3000 pg/mL.

PD-L2 protein may be detected by methods known to the art; for example ELISA, surface plasmon resonance (SPR) binding assays, quantitative fluorescent competition assays, and mass spectrometry methods.

In another aspect, serum PD-L2 protein levels may be estimated by quantitatively detecting and measuring serum PD-L2 mRNA, for example, using Quantitative RT-PCR (qRT-PCR).

The identification of metastatic tumor patients that will benefit from treatment with checkpoint inhibitors and benefit from immunooncology therapies generally has been particularly difficult. In particular identifying patients for whom durable responses are possible has been particularly difficult. E.g., Snyder et al., Genetic Basis for Clinical Response to CTLA-4 Blockade in Melanoma, NEJM 371:2189-2199 (2014).

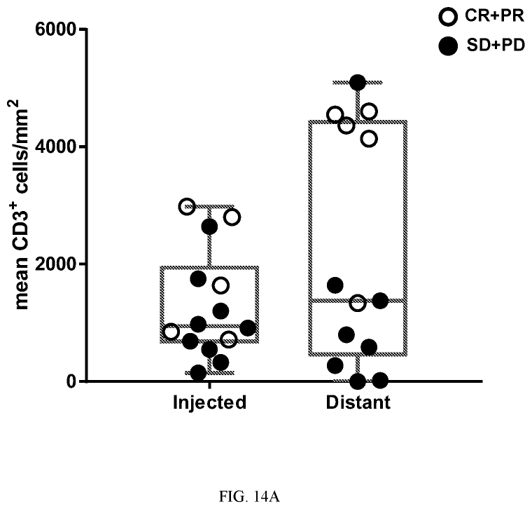

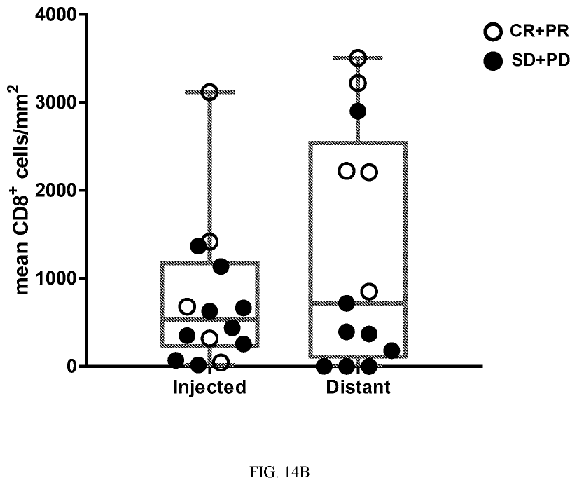

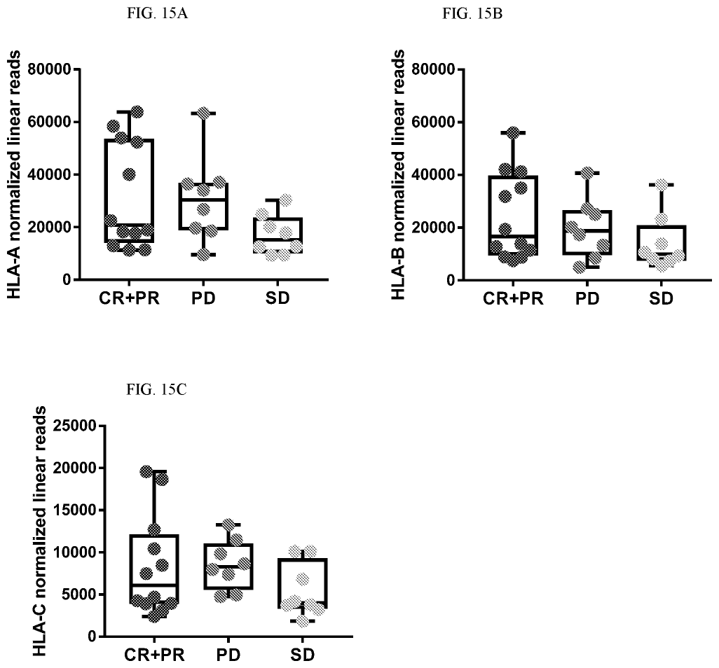

FIG. 14 shows that, surprisingly, the presence of T-cells in baseline tumors and the level of activation of T-cells in baseline tumor specimens does not correlate with response to immunooncology therapy. It is broadly believed that baseline TIL infiltration is a prognostic marker, with more infiltration correlating with better clinical outcomes. Gooden et al., The prognostic influence of tumour-infiltrating lymphocytes in cancer: a systematic review with meta-analysis, Br. J. Cancer 105:93-103 (2011). Surprisingly, clinical response, both in injected tumors (local) and non-injected tumors (remote), correlates with a high proportion of dendritic cells (DC) in baseline tumors. Further, surprisingly, clinical response, both in injected tumors (local) and non-injected tumors (remote), correlates with elevated serum levels of PD-L2. FIG. 9B shows the increased levels of serum PD-L2 in patients responding to intratumoral IMO-2125 in combination with systemic anti-CTLA4 treatment. Furthermore, tumors with a higher neutrophil score and greatly reduced mast cell score at baseline did not respond to combinatorial therapy, as shown in FIG. 9C.

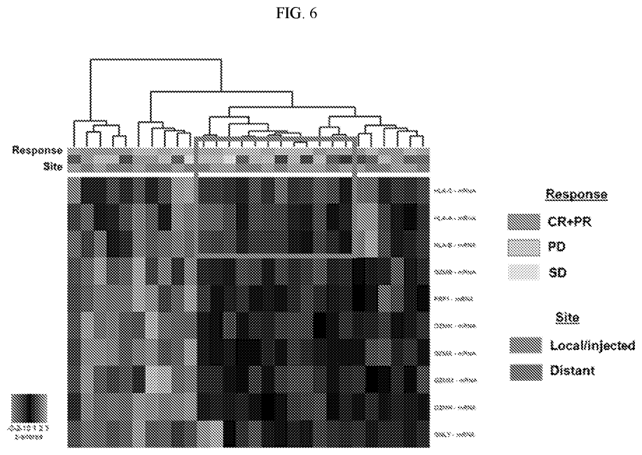

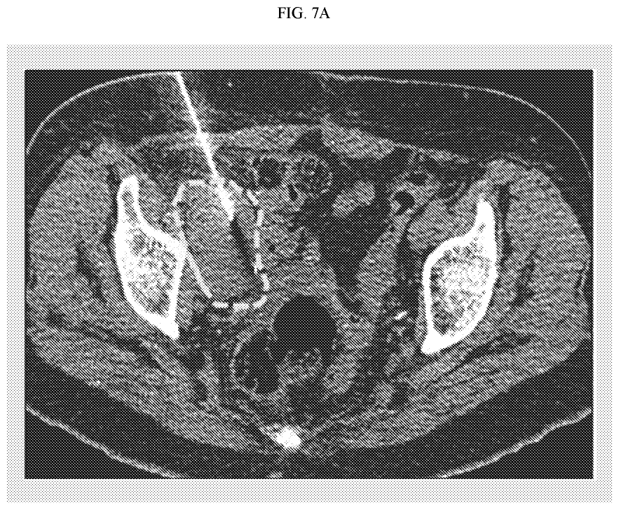

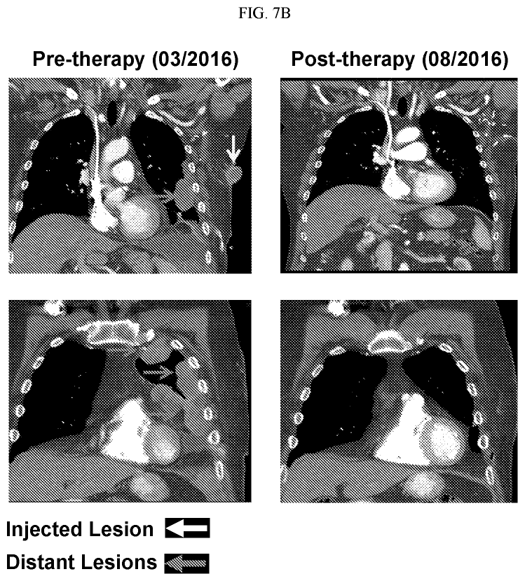

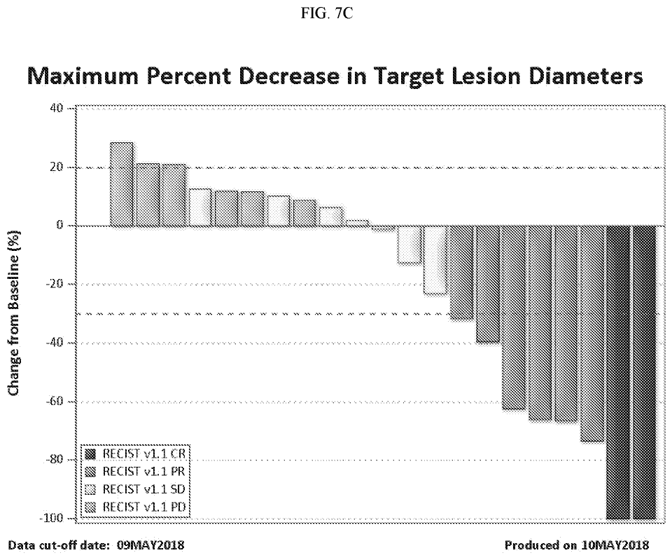

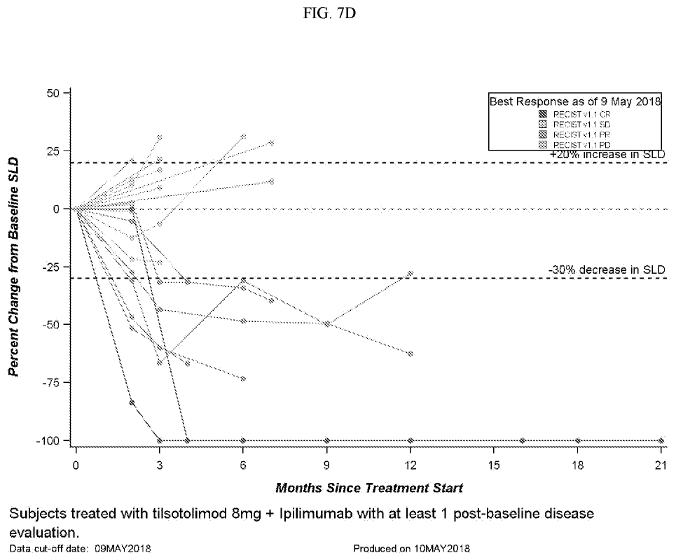

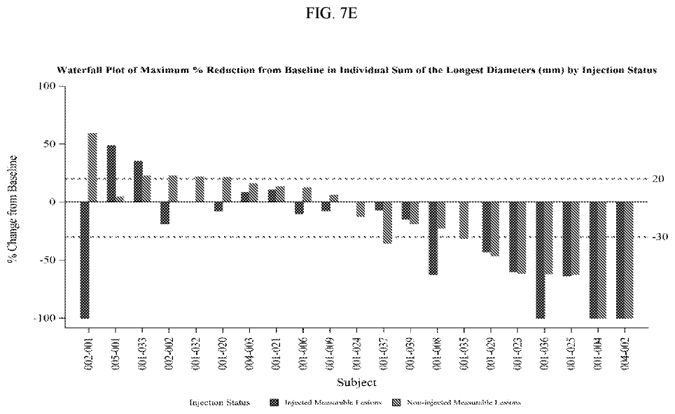

In one aspect, the TLR9 agonist is the oligonucleotide known as IMO-2125, which is described more fully herein, upon intratumoral injection induces global increases in expression of checkpoint genes, including IDO1 (5.3 fold), PDL1 (2.6 fold), PD1 (2.5 fold), IDO2 (5.9 fold), CEACAM1 (2.1 fold), OX40 (1.4 fold), TIM3 (2.9 fold), LAG3 (1.9 fold), CTLA4 (1.8 fold), and OX40L (1.5 fold). See FIG. 6B. By altering immune signaling in the tumor microenvironment, such changes in gene expression provide opportunities to improve responsiveness with checkpoint inhibitor therapy, and to achieve lasting anti-tumor immunity. Further, by targeting a single immune checkpoint molecule selected from the stronger inhibitory signals of PD-1 or CTLA-4, in connection with the robust activation of antigen presenting cells (e.g., DCs) and priming of T cells with IMO-2125, the invention provides the opportunity to balance anti-tumor responses with inhibitory signals, thereby also minimizing irAEs of checkpoint inhibitor therapy.

In another aspect, intratumoral administration of IMO-2125 in conjunction with systemic checkpoint inhibitor administration results in proliferation of T-cells in both treated tumors and untreated tumors. In another aspect, IT administration of IMO-2125 in conjunction with systemic ipilimumab administration results in T-cell proliferation in the IMO-2125 injected tumor and in remote tumors that have not been treated with IMO-2125. See Example 5.

In various embodiments, the patient has a cancer that was previously unresponsive to, or had become resistant to, a checkpoint inhibitor therapy. In some embodiments, the cancer is refractory or relapsed. For example, the cancer may be refractory or insufficiently responsive to an immunotherapy, such as anti-CTLA-4, anti-PD-1, or anti-PD-L1 and/or PD-L2 agent, including for example, one or more of ipilimumab, tremelimumab, pembrolizumab and nivolumab. In various embodiments, the cancer patient has progressed after or during treatment with an anti-CTLA-4, anti-PD-1, or anti-PD-L1 and/or PD-L2 agent, including for example, one or more of ipilimumab, tremelimumab, pembrolizumab and nivolumab (or agents related thereto) or shown no response to such treatment for at least about 12 weeks.

Other immune checkpoint inhibitors can be administered alone (e.g, in place of) or in combination with anti-CTLA4 or anti-PD-1/anti-PD-L1, such as an inhibitor of IDO (e.g., IDO-1 or IDO-2), LAG3, TIM3, among others. These and other immune checkpoint inhibitors are described in US 2016-0101128, which is hereby incorporated by reference in its entirety. For example, the patient may further receive a regimen of an IDO-1 inhibitor such as Epacadostat.

In various embodiments, the cancer is a primary cancer or a metastatic cancer. A primary cancer refers to cancer cells at an originating site that become clinically detectable, and may be a primary tumor. “Metastasis” refers to the spread of cancer from a primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. In some embodiments, the cancer is a relapsed or refractory cancer, for example, a sarcoma or a carcinoma.

The cancer may have an origin from any tissue. The cancer may originate from skin, colon, breast, or prostate, and thus may be made up of cells that were originally skin, colon, breast, or prostate, respectively. The cancer may also be a hematological malignancy, which may be lymphoma. In various embodiments, the primary or metastatic cancer is lung cancer, kidney cancer, prostate cancer, cervical cancer, colorectal cancer, colon cancer, pancreatic cancer, ovarian cancer, urothelial cancer, gastric/GEJ cancer, head and neck cancer, glioblastoma, Merkel cell cancer, head and neck squamous cell carcinoma (HNSCC), non-small cell lung carcinoma (NSCLC), small cell lung cancer (SCLC), bladder cancer, prostate cancer (e.g. hormone-refractory) and hematologic malignancies.

In some embodiments, the cancer is progressive, locally advanced, or metastatic carcinoma. In some embodiments, the cancer is metastatic melanoma, and may be recurrent. In some embodiments, the metastatic melanoma is stage III or IV, and may be stage IVA, IVB, or IVC. The metastasis may be regional or distant.

In various embodiments, the metastatic tumor is a low MHC Class I expressing tumor. In various embodiments, the low MHC Class I expressing tumor expresses less than 50% of normal MHC Class I mRNA expression. In some embodiments, the low MHC Class I expressing tumor expresses less than 35% of normal MHC Class I mRNA expression. In some embodiments, the low MHC Class I expressing tumor expresses less than 30% of normal MHC Class I mRNA expression. In some embodiments, the low MHC Class I expressing tumor expresses less than 25% of normal MHC Class I mRNA expression. In some embodiments, the low MHC Class I expressing tumor expresses no detectable levels of at least one MHC Class I mRNA.

In various embodiments, the metastatic tumor is a low MHC Class I expressing tumor. In various embodiments, the low MHC Class I expressing tumor expresses less than 50% of normal MHC Class I protein expression. In some embodiments, the low MHC Class I expressing tumor expresses less than 35% of normal MHC Class I protein expression. In some embodiments, the low MHC Class I expressing tumor expresses less than 30% of normal MHC Class I protein expression. In some embodiments, the low MHC Class I expressing tumor expresses less than 25% of normal MHC Class I protein expression. In some embodiments, the low MHC Class I expressing tumor expresses no detectable levels of at least one MHC Class I protein.

In various embodiments, the metastatic tumor has no measurable expression of B2M, the β2-microglobulin gene. In various embodiments, the B2M mRNA is detected, but there is no β2-microglobulin protein detected.

Gene expression of MHC Class I and B2M may be measured by any suitable technique in the art, such as, and without limitation, reverse transcriptase polymerase chain reaction (rtPCR) or quantitative PCR (qPCR), to detect mRNA presence or absence, or to quantitate mRNA expression level. Expression of MHC Class I proteins HLA-A, HLA-B, and HLA-C and β2-microglobulin protein may be measured by any suitable technique in the art, such as, and without limitation, immunohistochemistry staining of pretreatment tumor biopsy samples. Rodig et al., Sci. Transl. Med., “MHC proteins confer differential sensitivity to CTLA-4 and PD-1 blockade in untreated metastatic melanoma,” 10, eaar3342 (2018), discloses exemplary immunohistochemistry methods quantitating protein expression of each of the MHC Class I genes HLA-A, HLA-B, and HLA-C.

In some embodiments, patients are identified for treatment with methods of the invention by assessing the percentage of tumor cells in a tumor biopsy specimen for MHC Class I protein expression. In some embodiments, a patient with 50% or fewer tumor cells in a tumor biopsy expressing MHC Class I protein expression is treated. Rodig et al., Sci. Transl. Med., MHC proteins confer differential sensitivity to CTLA-4 and PD-1 blockade in untreated metastatic melanoma, 10, eaar3342 (2018), discloses exemplary methods to identify the percentage of tumor cells in a biopsy specimen expressing MHC Class I proteins.

In some embodiments, patients are identified for treatment with methods of the invention by assessing the expression level of the B2M gene. In some embodiments, patients with metastatic tumors expressing no detectable levels of B2M mRNA are selected for treatment.

IMO-2125 and related immunostimulatory oligonucleotides target TLR9, and act as TLR9 agonists to alter immune signaling in the tumor microenvironment, and induce anti-tumor T cell responses.

In accordance with various embodiments, the TLR9 agonist comprises at least two oligonucleotides linked together through their 3′ ends, so as to have multiple accessible 5′ ends. The linkage at the 3′ ends of the component oligonucleotides is independent of the other oligonucleotide linkages and may be directly via 3′ or 2′ hydroxyl groups, or indirectly, via a non-nucleotide linker or a nucleoside, utilizing either the 2′ or 3 ‘ hydroxyl positions of the nucleoside. Linkages may also employ a functionalized sugar or nucleobase of a 3’ terminal nucleotide. Exemplary TLR9 agonists are described in U.S. Pat. Nos. 8,420,615, 7,566,702, 7,498,425, 7,498,426, 7,405,285, 7,427,405, including Tables 1 and 2A-2D of each, the entire contents of which are hereby incorporated by reference in their entireties. Exemplary TLR9 agonists are also described in U.S. Pat. Nos. 7,745,606 and 8,158,768, the entire contents of which are hereby incorporated by reference in their entireties.

In various embodiments, the TLR agonist is selected from:

(SEQ ID NO: 1)
5′-TCTGACG₁TTCT-X-TCTTG₁CAGTCT-5′
(SEQ ID NO: 2)
5′-TCTGTCG₁TTCT-X-TCTTG₁CTGTCT-5′
(SEQ ID NO: 3)
5′-TCG₁TCG₁TTCTG-X-GTCTTG₁CTG₁CT-5′
(SEQ ID NO: 4)
5′-TCG₁AACG₁TTCG₁-X-G₁CTTG₁CAAG₁CT-5′
(SEQ ID NO: 5)
5′-CTGTCoG₂TTCTC-X-CTCTTG₂oCTGTC-5′
(SEQ ID NO: 6)
5′-CTGTCG₂TTCTCo-X-oCTCTTG₂CTGTC-5′
(SEQ ID NO: 7)
5′-TCG₁AACG₁TTCG₁-X-TCTTG₂CTGTCT-5′
(SEQ ID NO: 8)
5′-TCG₁AACG₁TTCG₁-Y-GACAG₁CTGTCT-5′
(SEQ ID NO: 9)
5′-CAGTCG₂TTCAG-X-GACTTG₂CTGAC-5′
(SEQ ID NO: 10)
5′-CAGTCG₁TTCAG-X-GACTTG₁CTGAC-5′
(SEQ ID NO: 11)
5′-TCG₁AACG₁TTCoG-Z-GoCTTG₁CAAG₁CT-5′
(SEQ ID NO: 12)
5′-TCG₁AACG₁TTCG₁-Y₂-TCTTG₁CTGTCTTG₁CT-5′
(SEQ ID NO: 13)
5′-TCG₁AACG₁TTCG₁-Y₂-TCTTG₁CTGUCT-5′
(SEQ ID NO: 14)
5′-TCG₁AACG₁ToTCoG-m-GoCToTG₁CAAG₁CT-5′
(SEQ ID NO: 15)
5′-TCG₁AACG₁TTCoG-Y₃-GACTTG₂CTGAC-5′
(SEQ ID NO: 16)
5′-TCG₁AACG₁TTCG₁-Y₄-TGTTG₁CTGTCTTG₁CT-5′
(SEQ ID NO: 17)
5′-TCG₂TCG₂TTU₁Y-M-YU₁TTG₂CTG₂CT-5′
(SEQ ID NO: 18)
5′-CAGTCG₂TTCAG-Y₃-TCTTG₁CTGTCT-5′
(SEQ ID NO: 19)
5′-TCG₁TACG₁TACG₁-X-G₁CATG₁CATG₁CT-5′
(SEQ ID NO: 20)
5′-TCG₁AACG₁TTCG-Z-GCTTG₁CAAG₁CT-5′
(SEQ ID NO: 21)
5′-TCG₁AACG₁TTCoG-Y₃-CTTG₂CTGACTTG₁CT-5′
(SEQ ID NO: 22)
5′-TCG₁AACG₁oTTCG₁-X₂-G₁CTToG₁CAAG₁CT-5′
(SEQ ID NO: 23)
5′-TCG₁AACG₁TTCG₁-Y₄-CATTG₁CTGTCTTG₁CT-5′
(SEQ ID NO: 24)
5′-TCG₁AACG₁TTCG₁-m-G₁CTTG₁CAAG₁CT-5′
(SEQ ID NO: 25)
5′-TCoG₁oAACoG₁TTCoG₁o-X₂-oG₁oCTTG₁oCAAoG₁oCT-5′
(SEQ ID NO: 26)
5′-ToCG₁oAACoG₁TTCoG₁o-X₂-oG₁oCTTG₁oCAAoG₁CoT-5′
(SEQ ID NO: 27)
5′-TCoG₁oAACoG₁TTCoG₁o-m-oG₁oCTTG₁oCAAoG₁oCT-5′
(SEQ ID NO: 28)
5′-TCoG₂oAACoG₂TTCoG₂o-X₂-oG₂oCTTG₂oCAAoG₂oCT-5′
(SEQ ID NO: 29)
5′-TCoG₁oAACoG₁TTCoGo-Z-oGoCTTG₁oCAAoG₁oCT-5′
and
(SEQ ID NO: 30)
5′-ToCG₁oAACoG₁TTCoGo-Z-oGoCTTG₁oCAAoG₁CoT-5′,

where G₁ is 2′-deoxy-7-deazaguanosine; G₂ is 2′-deoxy-arabinoguanosine; G, C, or U are 2′-O-methylribonucleotides; U₁ is 2′-deoxy-U; o is a phosphodiester linkage; X is a glycerol linker; X₂ is a isobutanetriol linker, Y is C3-linker; m is cis,trans-1,3,5-cyclohexanetriol linker; Y₂ is 1,3-propanediol linker; Y₃ is 1,4-butanediol linker; Y₄ is 1,5-pentandiol linker; Z is 1,3,5-pentanetriol linker; and M is cis,cis-1,3,5-cyclohexanetriol linker.

In various embodiments, the TLR9 agonist is selected from 5′-TCG₁AACG₁TTCG₁-X-G₁CTTG₁CAAG₁CT-5′ (SEQ ID NO:4), 5′-CTGTCoG₂TTCTC-X-CTCTTG₂oCTGTC-5′ (SEQ ID NO:5), 5′-CTGTCG₂TTCTCo-X-oCTCTTG₂CTGTC-5′ (SEQ ID NO:6), 5′-TCG₁AACG₁TTCG₁-Y-TCTTG₂CTGTCT-5′ (SEQ ID NO:7), and 5′-TCG₁AACG₁TTCG₁-Y-GACAG₁CTGTCT-5′ (SEQ ID NO:8), wherein X is a glycerol linker, Y is a C3-linker, G₁ is 2′-deoxy-7-deazaguanosine, G₂ is arabinoguanosine, and o is a phosphodiester linkage.

In various embodiments, the TLR9 agonist is 5′-TCG₁AACG₁TTCG₁-X-G₁CTTG₁CAAG₁CT-5′ (SEQ ID NO:4), wherein X is a glycerol linker and G₁ is 2′-deoxy-7-deazaguanosine, otherwise known as IMO-2125.

Alternative TLR9 agonists are immune stimulatory oligonucleotides disclosed in U.S. Pat. No. 8,871,732, which is hereby incorporated by reference in its entirety. Such agonists comprise a palindromic sequence of at least 8 nucleotides and at least one CG dinucleotide.

In accordance with embodiments of the invention, the immunostimulatory oligonucleotide (e.g., IMO-2125) is administered intratumorally. In some embodiments, the intratumoral administration is in a primary or secondary tumor (e.g., metastatic melanoma lesion). Intratumoral administration alters immune signaling in the tumor microenvironment, priming the immune system for an effective anti-tumor response, while inducing changes that are compatible with more effective checkpoint inhibitor therapy.

Illustrative dosage forms suitable for intratumoral administration include solutions, suspensions, dispersions, emulsions, and the like. The TLR9 agonist may be provided in the form of sterile solid compositions (e.g. lyophilized composition), which can be dissolved or suspended in sterile injectable medium immediately before use. They may contain, for example, suspending or dispersing agents known in the art.

In various embodiments, the TLR9 agonist is IMO-2125 and is administered intratumorally at from about 1 mg to about 20 mg, from about 4 mg to about 64 mg per dose, or in some embodiments from about 8 mg to about 64 mg per dose, or from about 12 mg to about 64 mg per dose, or from about 16 mg to about 64 mg per dose, or from about 20 mg to about 64 mg per dose. In some embodiments, IMO-2125 is administered at from about 20 mg to about 48 mg per dose, or about 20 mg to about 40 mg per dose. For example, in various embodiments, IMO-2125 is administered at about 4 mg, or about 8 mg, or about 12 mg, or about 16 mg, or about 20 mg, or about 24 mg, or about 28 mg, or about 32 mg, or about 36 mg, or about 40 mg, or about 44 mg, or about 48 mg, or about 52 mg, or about 56 mg, or about 60 mg, or about 64 mg per dose, e.g. intratumorally.

In various embodiments, about 1, about 2, or about 3 to about 12 doses of the TLR9 agonist (e.g. IMO-2125) are administered (e.g. about 1 dose, or about 2 doses, or about 3 doses, or about 4 doses, or about 5 doses, or about 6 doses, or about 7 doses, or about 8 doses, or about 9 doses, or about 10 doses, or about 11 doses, or about 12 doses). In various embodiments, about 4 to about 8 doses are administered over 10 to 12 weeks. In some embodiments, about 6 doses are administered over 10 to 12 weeks. In some embodiments, therapy is initiated with 3 to 5 weekly doses of IMO-2125, optionally followed by 3 to 8 maintenance doses, which are administered about every three weeks. In some embodiments, an IMO-2125 dose is administered in weeks 1, 2, 3, 5, 8, and 11. The IMO-2125 doses may be administered in the same or different lesions.

During the regimen of IMO-2125 (or other TLR9 agonist), one or more checkpoint inhibitor therapies are administered to take advantage of the changes in immune signaling. The one or more checkpoint inhibitors can be administered parenterally, including intravenously, intratumorally, or subcutaneously, among other methods. In some embodiments, the patient receives an anti-CTLA-4 agent. For example, the anti-CTLA-4 agent may be an antibody that targets CTLA-4, for instance an antagonistic antibody. In various embodiments, the anti-CTLA-4 is ipilimumab (e.g. YERVOY, BMS-734016, MDX-010, MDX-101). In various embodiments, the anti-CTLA-4 is tremelimumab (e.g. CP-675,206, MEDIMMUNE). In other embodiments, the immunotherapy agent is an anti-PD-1 agent. For example, the anti-PD-1 agent may be an antibody that targets the PD-1, for instance, inhibiting the interaction between PD-1 and PD-L1 (and/or PD-L2). In various embodiments, the anti-PD-1 agent is nivolumab (ONO-4538/BMS-936558, MDX1106 or OPDIVO). In various embodiments, the anti-PD-1 agent is pembrolizumab (KEYTRUDA or MK-3475). In various embodiments, the anti-PD-1 agent is pidilizumab (CT-011 or MEDIVATION).

In some embodiments, the present immunotherapy agent is an anti-PD-L1 and/or PD-L2 agent. For example, in various embodiments, the anti-PD-L1 and/or PD-L2 agent is an antibody that targets PD-L1 and/or PD-L2, for instance, inhibiting the interaction between PD-1 and PD-L1 and/or PD-L2. In various embodiments, the anti-PD-L1 and/or PD-L2 agent is atezolizumab (TECENTRIQ, ROCHE) BMS 936559 (BRISTOL MYERS SQUIBB), or MPDL328OA (ROCHE).

In various embodiments, the anti-CTLA-4, anti-PD-1, or anti-PD-L1 and/or PD-L2 agent (e.g. YERVOY, OPDIVO, or KEYTRUDA, or comparable agents thereto) is administered at a dose of about 1 mg/kg, or about 2 mg/kg, or about 3 mg/kg, or about 4 mg/kg, or about 5 mg/kg, e.g. intravenously. For example, in some embodiments, the dose of an anti-CTLA-4 agent, e.g. YERVOY, is about 3 mg/kg. For example, in some embodiments, the dose of an anti-PD-1 agent, e.g. OPDIVO, is about 3 mg/kg. For example, in some embodiments, the dose of an anti-PD-1 agent, e.g. KEYTRUDA, is about 2 mg/kg. In various embodiments, the initial dose of the anti-CTLA-4, anti-PD-1, or anti-PD-L1 and/or PD-L2 agent (e.g. YERVOY, OPDIVO, or KEYTRUDA, or comparable agents thereto) is administered at least one week after the initial TLR9 agonist dose, for example in about weeks 2, 3 or 4.

In some embodiments, the immunotherapy agent is anti-CTLA-4 (e.g. YERVOY), anti-PD-1 (e.g. OPDIVO or KEYTRUDA), or anti-PD-L1 and/or anti-PD-L2 agent, which is administered from about 2 to about 6 times (e.g. about 2 times, or about 3 times, or about 4 times, or about 5 times, or about 6 times). In some embodiments, the immunotherapy agent, e.g. anti-CTLA-4 (e.g. YERVOY), anti-PD-1 (e.g. OPDIVO or KEYTRUDA), or anti-PD-L1 and/or PD-L2 agent is administered about 4 times.

In some embodiments, the immunotherapy agent is an anti-CTLA-4 agent such as YERVOY and is dosed at 3 mg/kg i.v. over about 90 minutes about every 3 weeks. In some embodiments, the immunotherapy agent is an anti-PD-1 agent such as OPDIVO and is dosed at about 3 mg/kg i.v. over about 60 minutes about every 2 weeks. In some embodiments, the immunotherapy agent is an anti-PD-1 agent such as KEYTRUDA and is dosed at about 2 mg/kg i.v. over about 30 minutes about every 3 weeks.

In some embodiments, maintenance doses of the TLR9 agonist (e.g. IMO-2125), along with dosing of anti-CTLA-4, anti-PD-1, or anti-PD-L1 and/or PD-L2 agent (e.g. YERVOY, OPDIVO, or KEYTRUDA, or comparable agents thereto) are administered about every 3 weeks.

In various embodiments, the present immunostimulatory oligonucleotides allow for a dose reduction of the immunotherapy to about 10%, or about 20%, or about 30%, or about 40%, or about 50%, or about 60%, or about 70%, or about 80%, or about 90%, or about 100% of a monotherapy dose. For example, in some embodiments, an immunotherapy dose is about 0.1 mg/kg, or about 0.3 mg/kg, or about 0.5 mg/kg, or about 0.7 mg/kg, or about 1 mg/kg, or about 1.5 mg/kg, or about 2 mg/kg, or about 2.5 mg/kg, or about 3 mg/kg.

In some embodiments, IMO-2125 is administered intratumorally to a metastatic melanoma patient previously found to be unresponsive or only partially responsive to PD-1 blockade therapy. IMO-2125 is administered at a dose of from 4 to 32 mg per dose (e.g., about 16 mg, about 20 mg, about 24 mg, about 28 mg, or about 32 mg) in weeks 1, 2, 3, 5, 8, and 11, with ipilimumab i.v. at 3 mg/kg. Ipilimumab can be administered every three weeks, beginning in week 2 (e.g., weeks 2, 5, 8, and 11). Alternatively, pembrolizumab can be administered i.v. at 2 mg/kg every three weeks beginning on week 2 (e.g., weeks 2, 5, 8, and 11).

In some embodiments, the patient further receives a regimen of Epacadostat (an IDO-1 inhibitor), which may be administered at from 25 mg to 300 mg orally, about twice daily. The regimen may be administered for about 5 day cycles. The first dose of Epacadostat may be administered starting at about one week following the initial IMO-2125 (or other TLR9 agonist) intratumoral injection.

In various embodiments, without wishing to be bound by theory, the invention provides for a more balanced immune response in a cancer patient, including cancer patients with advanced, metastatic disease. The combination therapy described herein can eliminate or reduce deficiencies that are observed in the respective monotherapies. For example, various patients are refractory to immunotherapies, or such monotherapies are hampered by extensive side effect profiles. Further as the field is moving to combinations of immunotherapies (e.g. YERVOY and OPDIVO), such side effects are likely to be more problematic.

In various embodiments, the combination therapy allows for activation and/or maturation of dendritic cells, e.g. plasmacytoid dendritic cells, and modulates the tumor microenvironment (TME) in both treated and distant tumors. For example, in various embodiments, the combination therapy provides for improvements in the amount or quality of TILs and/or CD8⁺ T cells to promote anti-tumor activities. For example, primed T cells are observed to invade both the proximal and distal tumors. Such primed T cells are suited for tumor invasion, particularly at distal sites (e.g. secondary tumors), and, without wishing to be bound by theory, encounter a tumor environment that has reduced tolerance mechanisms in place. In various embodiments, the combination therapy provides for stimulation of interferons (e.g. IFN-α) and various Th1 type cytokines (e.g. IFN-γ, IL-2, IL-12, and TNF-β). See Example 4.

The invention provides, in various embodiments, methods for treating cancers, including metastatic cancers, in which the overall host immune milieu is reengineered away from tumor tolerance. For example, a local TME is created that both disrupts pathways of immune tolerance and suppression and allow for tumor regression. The present methods provide in some embodiments, a TME capable of propagating a robust immune response.

In various embodiments, a cancer patient's DCs are immature and unable to take up, process, or present antigens. These DCs may also be inhibited from migrating to regional lymph nodes or may induce tolerance, especially when presenting self-antigens. The cancer patient's tumor site may also be infiltrated with regulatory T cells that are able to mediate suppression of antigen-primed T cells. The helper CD4 T cell response may also be skewed toward a Th2 phenotype, which inhibits the initiation of Th1 T cells and effective cellular immunity. The tumor cells may express aberrant MHC class I molecules or β2-microglobulin, resulting in inadequate antigen presentation and, thus, inefficient recognition of tumors by effector T cells. Finally, tumor cells and the surrounding stroma may release a number of suppressive cytokines, such as IL-6, IL-10, and TGF-β. This creates an environment that is not conducive to local immunity, which allows tumor cells to escape. In various embodiments, the present methods allow for an environment that is conducive to local immunity against tumors, e.g., without limitation, maturation of DCs and/or reduction of regulatory T cells and Th2 CD4 T cells.

In some embodiments, the combination therapy according to the invention alters the balance of immune cells in favor of immune attack of a tumor. For instance, in some embodiments, the present methods shift the ratio of immune cells at a site of clinical importance, e.g. at the site of agent administration or a distal site, in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, the present methods increase a ratio of effector T cells to regulatory T cells. In various embodiments, this altered balance of immune cells is affected locally/proximally and/or systemically/distally. In various embodiments, this altered balance of immune cells is affected in the TME.

Further, in various embodiments, the present methods allow for a robust anti-tumor immune response that does not come at the expense of significant side effects (e.g., irAEs), e.g. relative to side effects observed when one or more immunotherapies are used in the absence of the TLR9 agonist.

For example, the combination therapy reduces one or more side effects of an immunotherapy, e.g. an anti-CTLA-4, anti-PD-1, or anti-PD-L1 and/or PD-L2 agent, including for example, one or more of YERVOY, OPDIVO, and KEYTRUDA or agents related thereto. Such side effects include: fatigue, cough, nausea, loss of appetite, skin rash, itching pruritus, rash, and colitis. In some embodiments, the side effects are intestinal problems (e.g. colitis) that can cause perforations in the intestines. Signs and symptoms of the colitis may include: diarrhea or more bowel movements than usual; blood in the stools or dark, tarry, sticky stools; and abdominal pain or tenderness. In some embodiments, the side effects are liver problems (e.g. hepatitis) that can lead to liver failure. Signs and symptoms of hepatitis may include: yellowing of skin or the whites of the eyes; dark urine; nausea or vomiting; pain on the right side of the stomach; and bleeding or bruising more easily than normal. In some embodiments, the side effects are skin problems that can lead to severe skin reactions. Signs and symptoms of severe skin reactions may include: skin rash with or without itching; sores in the mouth; and the skin blisters and/or peels. In some embodiments, the side effects are nerve problems that can lead to paralysis. Symptoms of nerve problems may include: unusual weakness of legs, arms, or face; and numbness or tingling in hands or feet. In some embodiments, the side effects are hormone gland problems (e.g. pituitary, adrenal, and thyroid glands). Signs and symptoms include: persistent or unusual headaches; unusual sluggishness; feeling cold all the time; weight gain; changes in mood or behavior such as decreased sex drive, irritability, or forgetfulness; and dizziness or fainting. In some embodiments, the side effects are ocular problems. Symptoms may include: blurry vision, double vision, or other vision problems; and eye pain or redness.

In some embodiments, patients experience fewer incidences of colitis, crohn's disease, or other GI involved irAE in accordance with the present invention.

In some embodiments, the patient achieves longer progression-free interval or longer survival (e.g., as compared to monotherapy), or in some embodiments, achieves remission or complete response. A complete response refers to the disappearance of all signs of cancer in response to treatment.

This invention is further illustrated by the following non-limiting examples.

### EXAMPLES

### Example 1 Identification of Patients Likely to Respond to Tilsotolimod in Combination with Ipilimumab

Fresh metastatic melanoma tumor tissue was disaggregated to generate a single cell suspension for staining. PBMCs were thawed, washed and resuspended for staining. Surface staining was performed in FACS Wash Buffer (1×DPBS with 1% Bovine Serum Albumin) for 30 min on ice using fluorochrome-conjugated monoclonal antibodies from BD Biosciences, Biolegend, or eBioscience, as described previously. Cells were fixed in 1% paraformaldehyde solution for 20 minutes at room temperature following surface staining. For panels containing transcription factors, cells were fixed and permeabilized using the eBioscience FoxP3 kit according to the manufacturer's instructions. Samples were acquired using the BD FACSCanto II or BD Fortessa X20 and analyzed using FlowJo Software v 7.6.5 (Tree Star). Dead cells were stained using AQUA live/dead dye (Invitrogen) and excluded from the analysis.

RNA was extracted from core needle biopsies preserved in RNA later using the Qiagen AllPrep Universal Kit (Cat #80224) according to the manufacturers' instructions. Purity and concentration were assessed using Nanodrop. RNA was assayed using the Nanostring Pan Cancer Immune Panel and analyzed using the nSolver Advanced Analysis Software.

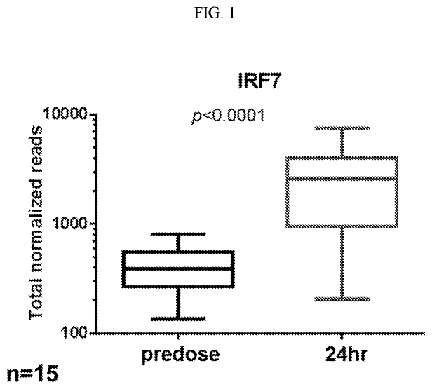

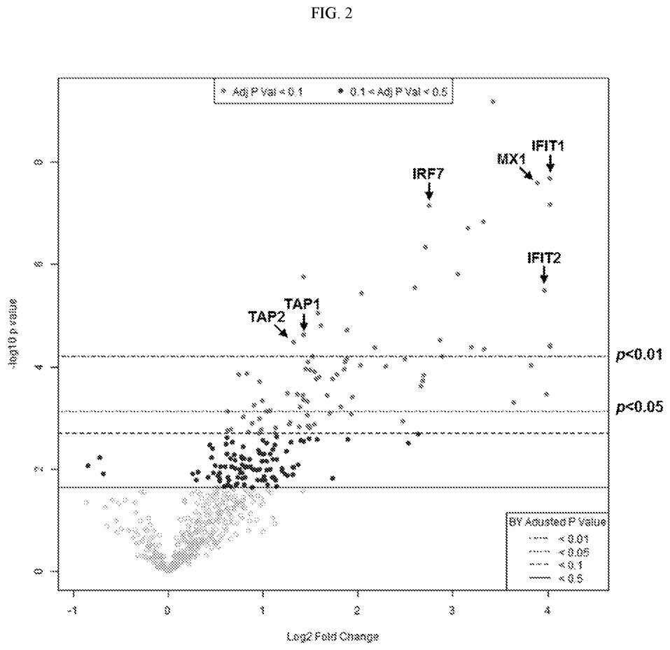

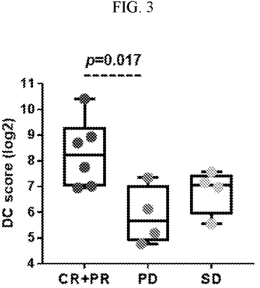

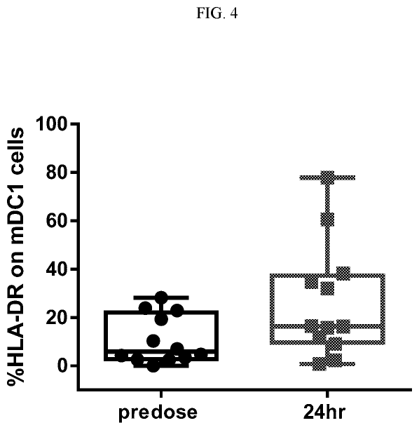

Gene expression profiling of tumor tissue collected from the injected tumor lesion at baseline and the same lesion 24 hours post injection demonstrated that intratumoral tilsotolimod triggered activation of a Type-I interferon response profile. FIG. 1 shows the induction of a key gene in this pathway, IRF-7. The volcano plot shown in FIG. 2 demonstrates that several Type-I IFN pathway genes that are elevated and indicates the level of significance (p<0.01). In addition, the presence of a dendritic cell expression profile (DC score) was found to be higher prior to therapy in patients that subsequently respond (FIG. 3, p<0.017) and local DCs were found to have gained the maturation marker HLA-DR (MHC class II) in some patients after i.t. tilsotolimod (FIG. 4, p=0.07) indicating that this drug was able induce maturation of local DCs which in turn have better antigen presentation capacity and could induce a more favorable environment for subsequent T cell activation.

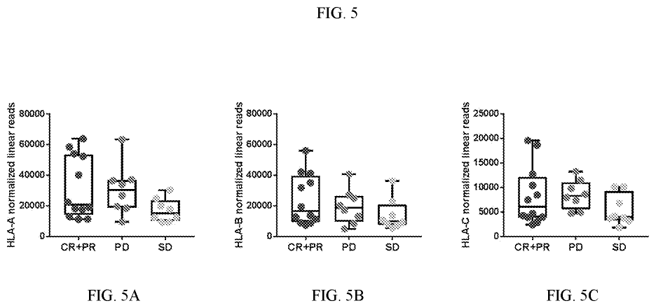

Unexpectedly, this combination treatment was able to overcome a known mechanism of resistance to single agent ipilimumab which has been linked to the need for high levels of the antigen presentation molecule MHC class I which is comprised of three major genes (HLA-A, HLA-B, HLA-C). Gene expression profiling of baseline tumor biopsies revealed that the combination of tilsotolimod and ipilimumab is able to overcome this mechanism of resistance as some responding patients had a lower expression level of HLA-A, HLA-B, HLA-C. FIG. 5 shows the gene expression profile of each gene individually and FIG. 6 shows the cumulative expression of all the genes in this cytotoxicity signature. The observed clinical data are surprising and unexpected because the expectation of one skilled in the art is that normal or higher levels of MHC Class I expression is required for response to immune checkpoint inhibitors.

### Example 2 Clinical Stratification of Patients Based on MHC Class I Expression

A pretreatment biopsy sample is collected from a patient with metastatic melanoma. The sample is sectioned and the sections fixed for chromogenic immunohistochemistry (IHC). A dual IHC for MHC class I (HLA-A, HLA-B, and HLA-C, clone EMR8-5, 1:6000; Abcam) is used to identify cells in the section expressing a MHC Class I protein. MHC class I, MHC class II, and (32M staining is scored for the percentage of malignant cells in 10% increments (0 to 100%) with positive membrane staining within the entire tissue section, as determined by the consensus of two pathologists. The result of the visual analysis is a percentage of malignant cells expressing a MHC Class I protein.

Patients whose tumors comprise less than 50% malignant cells expressing a MHC Class I protein are selected preferentially for tilsotolimod co-administered with ipilimumab.

### Example 3 Clinical Stratification of Patients Based on Dendritic Cell Enrichment in Baseline Tumor Specimens

A pretreatment biopsy sample is collected from a patient with metastatic melanoma. The sample is disaggregated and the myeloid cells separated from the bulk specimen. Fresh tumor tissue is disaggregated using a medimachine followed by filtering to generate a single cell suspension. Flow cytometry is used to identify live cells that possess one or more dendritic cell surface markers, for example, CD1c, CD11c, CD141, and CD141. The number of live dendritic cells per 100,000 cells is determined. This value is compared to the number of live dendritic cells per 100,000 cells in the patient's peripheral blood mononuclear cells (circulating DC level). The tumor biopsy is considered enriched for dendritic cells if the tumor DCs are 8% or more above the circulating DC level. Patients with tumor biopsy specimens enriched for DCs are selected for treatment with IMO-2125 in combination with an immune checkpoint inhibitor.

### Example 4 Intratumoral Administration of IMO-2125 Stimulates Type 1 Interferon Response

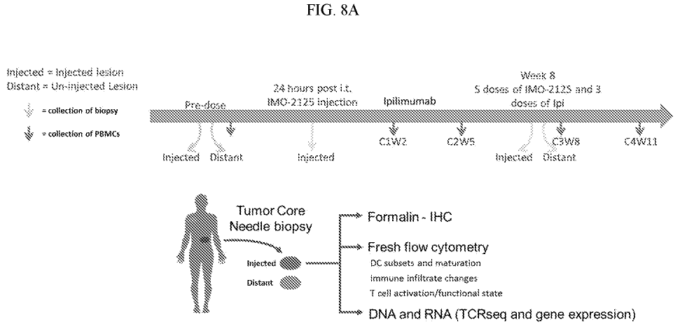

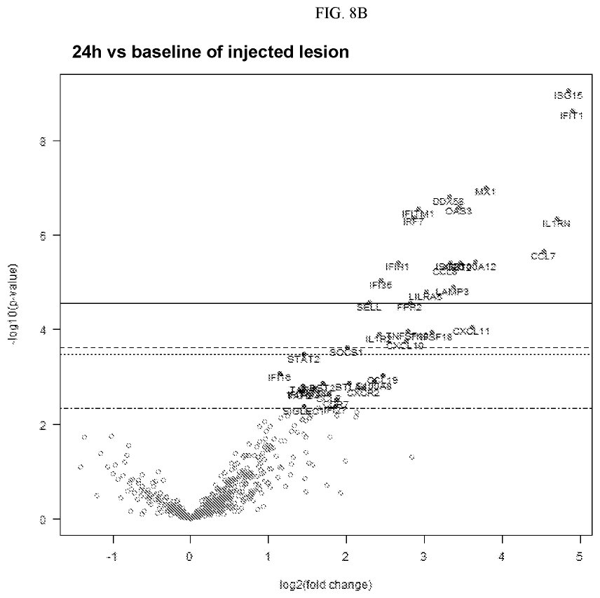

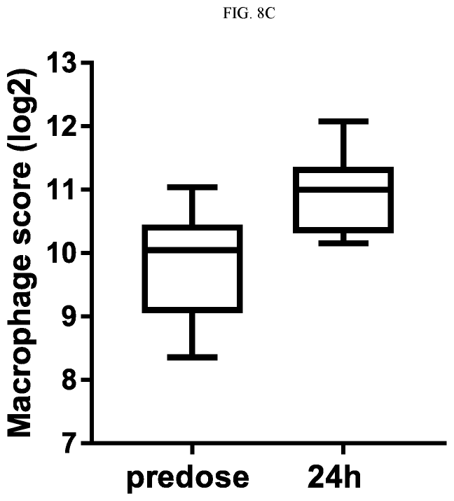

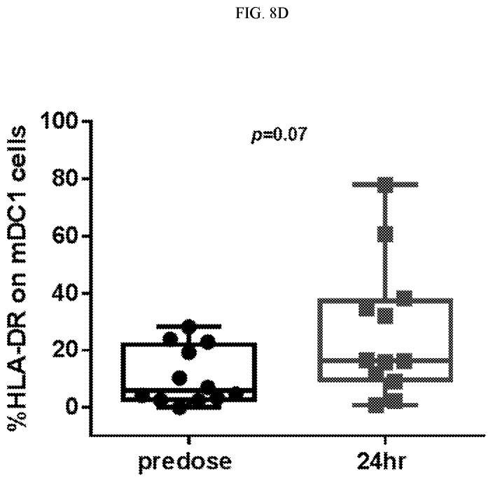

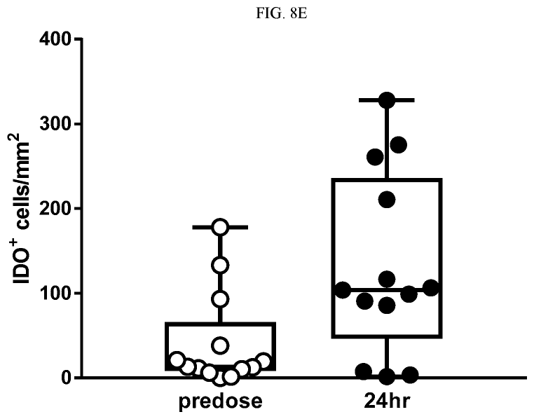

As depicted in FIG. 8A, tumor tissue and peripheral blood were collected from patients participating in the NCT02644967 clinical trial. To determine the impact of intratumoral administration of IMO-2125 (tilsotolimod) on the injected tumor, tumor tissue was collected at baseline, before IMO-2125 administration, and 24-hours after administration. The gene expression profile was determined for each tumor sample using NanoString gene profiling.

RNA was extracted from core needle biopsies preserved in RNAlater using the Qiagen AllPrep Universal Kit (catalog #80224) according to the manufacturer's instructions. Purity and concentration of the resulting RNA preparation was assessed using Nanodrop. RNA was assayed using the Nanostring Pan Cancer Immune Panel and the resulting data analyzed using the nSolver Advanced Analysis Software.

FIG. 8B shows the comparison of the baseline gene expression compared to the 24-hour post-injection gene expression profile. Intratumoral injection of IMO-2125 induces a type 1 interferon response, illustrated by the significant upregulation of IRF7, IL12A, IL1RN, CCL8, and CCL8 (adjusted p<0.01) genes. The upregulated gene profile includes both type I and type II interferon (IFNγ) response, e.g. IDO and PD-L1 (CD274), but did not result in the upregulation of “classical” IFNγ genes such as the MHC Class I genes or IRF1.

Markers of DC activation, for example, CD80 and IL12, and chemoattractants CCL7 and CCL8 were found to be upregulated at the 24-hour sampling time after IMO-2125 administration. See Table 1 below. These data correlate with the increase in the macrophage gene expression score (CD163, CD68, CD84, MS4A4A) (p=0.0003, n=12 paired samples) as depicted in FIG. 8C. Maturation of the CD1c+ subset is further demonstrated by the upregulation of MHC class II (HLA-DR) in a subset of patients as detected by flow cytometry on fresh tumor tissue (p=0.07, n=12; shown in FIG. 8D). Also, IDO expression was induced by IMO-2125 administration as detected by IHC and RNA expression (p=0.0012; n=13, shown in FIG. 8E).

Table 1 shows the top 70 enriched mRNAs of the 600 measured, sorted by p-value.

| | Log2 | std | Linear | | | |
|---|---|---|---|---|---|---|
| | fold | error | fold | | BY p- | |
| Gene Name | change | (log2) | change | p-value | value | probe.ID |
| ISG15-mRNA | 4.85 | 0.476 | 28.8 | 8.81E−10 | 3.68E−06 | NM_005101.3:305 |
| IFIT1-mRNA | 4.9 | 0.508 | 29.9 | 2.32E−09 | 4.85E−06 | NM_001548.3:1440 |
| MX1-mRNA | 3.79 | 0.489 | 13.8 | 9.76E−08 | 0.000136 | NM_002462.2:1485 |
| DDX58-mRNA | 3.32 | 0.441 | 10 | 1.54E−07 | 0.000161 | NM_014314.3:2130 |
| OAS3-mRNA | 3.45 | 0.472 | 11 | 2.48E−07 | 0.000196 | NM_006187.2:4980 |
| IFITM1-mRNA | 2.93 | 0.403 | 7.62 | 2.81E−07 | 0.000196 | NM_003641.3:482 |
| IRF7-mRNA | 2.87 | 0.405 | 7.29 | 4.16E−07 | 0.000234 | NM_001572.3:1763 |
| IL1RN-mRNA | 4.71 | 0.668 | 26.2 | 4.49E−07 | 0.000234 | NM_000577.3:480 |
| CCL7-mRNA | 4.53 | 0.714 | 23.1 | 2.21E−06 | 0.00103 | NM_006273.2:120 |
| S100A12-mRNA | 3.65 | 0.597 | 12.6 | 3.71E−06 | 0.00125 | NM_005621.1:260 |
| ISG20-mRNA | 3.33 | 0.545 | 10 | 3.81E−06 | 0.00125 | NM_002201.4:358 |
| IFIH1-mRNA | 2.67 | 0.437 | 6.37 | 3.81E−06 | 0.00125 | NM_022168.2:185 |
| IFIT2-mRNA | 3.47 | 0.569 | 11.1 | 3.89E−06 | 0.00125 | NM_001547.4:1995 |
| CCL8-mRNA | 3.27 | 0.544 | 9.68 | 4.68E−06 | 0.0014 | NM_005623.2:689 |
| IFI35-mRNA | 2.44 | 0.427 | 5.44 | 9.17E−06 | 0.00255 | NM_005533.3:415 |
| LAMP3-mRNA | 3.37 | 0.603 | 10.3 | 1.27E−05 | 0.00331 | NM_014398.3:1400 |
| LILRA5-mRNA | 3.02 | 0.55 | 8.12 | 1.60E−05 | 0.00393 | NM_181879.2:545 |
| SELL-mRNA | 2.29 | 0.434 | 4.9 | 2.68E−05 | 0.00602 | NR_029467.1:1585 |
| FPR2-mRNA | 2.82 | 0.535 | 7.06 | 2.74E−05 | 0.00602 | NM_001462.3:1200 |
| CXCL11-mRNA | 3.62 | 0.756 | 12.3 | 8.74E−05 | 0.0183 | NM_005409.4:282 |
| TNFSF10-mRNA | 2.79 | 0.592 | 6.9 | 0.000108 | 0.0215 | NM_003810.2:115 |
| TNFSF18-mRNA | 3.1 | 0.655 | 8.56 | 0.000114 | 0.0216 | NM_005092.2:175 |
| IL1R2-mRNA | 2.42 | 0.515 | 5.34 | 0.000125 | 0.0227 | NM_173343.1:113 |
| CXCL10-mRNA | 2.76 | 0.613 | 6.79 | 0.000174 | 0.0304 | NM_001565.1:40 |
| SOCS1-mRNA | 2.01 | 0.459 | 4.03 | 0.000242 | 0.0404 | NM_003745.1:1025 |
| STAT2-mRNA | 1.45 | 0.342 | 2.73 | 0.000333 | 0.0535 | NM_005419.2:1965 |
| IFI16-mRNA | 1.15 | 0.297 | 2.21 | 0.000841 | 0.13 | NM_005531.1:2255 |
| CCL19-mRNA | 2.47 | 0.647 | 5.55 | 0.000928 | 0.138 | NM_006274.2:401 |
| S100A8-mRNA | 2.36 | 0.636 | 5.12 | 0.00124 | 0.179 | NM_002964.3:115 |
| BTLA-mRNA | 2.03 | 0.554 | 4.09 | 0.00134 | 0.185 | NM_181780.2:305 |
| BST2-mRNA | 1.7 | 0.464 | 3.25 | 0.00137 | 0.185 | NM_004335.2:560 |
| TAP1-mRNA | 1.44 | 0.397 | 2.71 | 0.00154 | 0.201 | NM_000593.5:2075 |
| CD38-mRNA | 1.59 | 0.446 | 3.01 | 0.00173 | 0.213 | NM_001775.2:460 |
| CXCR2-mRNA | 2.22 | 0.618 | 4.65 | 0.00174 | 0.213 | NM_001557.2:2055 |
| IFITM2-mRNA | 1.45 | 0.412 | 2.73 | 0.00191 | 0.228 | NM_006435.2:390 |
| TAP2-mRNA | 1.41 | 0.403 | 2.67 | 0.00197 | 0.229 | NM_000544.3:909 |
| CCL2-mRNA | 1.77 | 0.515 | 3.42 | 0.00232 | 0.262 | NM_002982.3:123 |
| CCR7-mRNA | 1.88 | 0.56 | 3.68 | 0.00303 | 0.333 | NM_001838.2:1610 |
| IFI27-mRNA | 1.86 | 0.581 | 3.62 | 0.00414 | 0.438 | NM_005532.3:390 |
| SIGLEC1-mRNA | 1.45 | 0.453 | 2.73 | 0.00419 | 0.438 | NM_023068.3:5165 |
| LAG3-mRNA | 1.64 | 0.521 | 3.12 | 0.00468 | 0.477 | NM_002286.5:1735 |
| CCR1-mRNA | 1.49 | 0.484 | 2.82 | 0.00542 | 0.539 | NM_001295.2:535 |
| PPBP-mRNA | 2.13 | 0.692 | 4.37 | 0.00559 | 0.543 | NM_002704.2:330 |
| CCL13-mRNA | 1.54 | 0.509 | 2.9 | 0.00632 | 0.6 | NM_005408.2:320 |
| PTGS2-mRNA | 1.87 | 0.623 | 3.66 | 0.0067 | 0.615 | NM_000963.1:495 |
| CD80-mRNA | 1.25 | 0.418 | 2.38 | 0.00677 | 0.615 | NM_005191.3:1288 |
| IDO1-mRNA | 2.12 | 0.714 | 4.35 | 0.00712 | 0.633 | NM_002164.3:50 |
| CD274-mRNA | 1.53 | 0.517 | 2.88 | 0.00741 | 0.636 | NM_014143.3:1245 |
| STAT1-mRNA | 1.16 | 0.392 | 2.23 | 0.00747 | 0.636 | NM_007315.2:205 |
| CD1D-mRNA | 1.44 | 0.489 | 2.72 | 0.00767 | 0.641 | NM_001766.3:1428 |
| LILRB2-mRNA | 1.43 | 0.491 | 2.69 | 0.00815 | 0.668 | NM_005874.1:595 |
| NOD2-mRNA | 1.48 | 0.51 | 2.78 | 0.00841 | 0.676 | NM_022162.1:4080 |
| STAT4-mRNA | 0.949 | 0.33 | 1.93 | 0.00869 | 0.685 | NM_003151.2:789 |
| TNFSF13B-mRNA | 1.33 | 0.479 | 2.52 | 0.0108 | 0.834 | NM_006573.4:1430 |
| CD48-mRNA | 1.02 | 0.367 | 2.03 | 0.0111 | 0.837 | NM_001778.2:270 |
| CD47-mRNA | 0.881 | 0.318 | 1.84 | 0.0112 | 0.837 | NM_001777.3:897 |
| CCND3-mRNA | 0.978 | 0.367 | 1.97 | 0.0142 | 1 | NM_001760.2:1215 |
| IL15RA-mRNA | 1.29 | 0.486 | 2.45 | 0.0144 | 1 | NM_002189.2:505 |
| IL12A-mRNA | 1.37 | 0.515 | 2.58 | 0.0149 | 1 | NM_000882.2:775 |
| TLR3-mRNA | 1.45 | 0.558 | 2.72 | 0.0167 | 1 | NM_003265.2:230 |
| TFRC-mRNA | −1.02 | 0.4 | 0.493 | 0.0181 | 1 | NM_003234.1:1220 |
| CXCL13-mRNA | 1.48 | 0.582 | 2.78 | 0.0187 | 1 | NM_006419.2:210 |
| MRC1-mRNA | −1.37 | 0.541 | 0.387 | 0.019 | 1 | NM_002438.2:525 |
| CCL11-mRNA | 1.79 | 0.705 | 3.45 | 0.0192 | 1 | NM_002986.2:378 |
| LILRB1-mRNA | 1.07 | 0.431 | 2.11 | 0.0206 | 1 | NM_001081637.1:2332 |
| IRF2-mRNA | 0.957 | 0.384 | 1.94 | 0.0206 | 1 | NM_002199.3:1624 |
| CSF2RB-mRNA | 1.21 | 0.496 | 2.32 | 0.0229 | 1 | NM_000395.2:3300 |
| GZMB-mRNA | 1.16 | 0.474 | 2.23 | 0.0232 | 1 | NM_004131.3:540 |
| PSMB8-mRNA | 0.884 | 0.364 | 1.85 | 0.0239 | 1 | NM_004159.4:1215 |

### Example 5 Combination IT IMO-2125 and Systemic CPI Therapy Induces Local and Remote T-Cell Proliferation

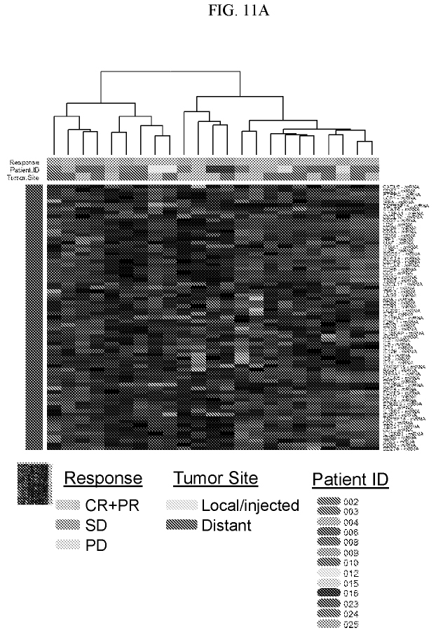

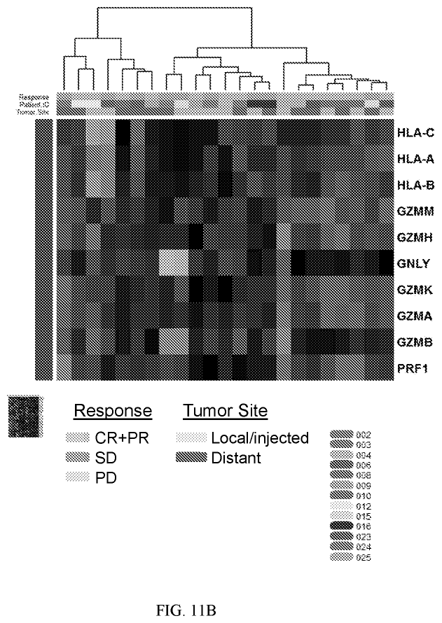

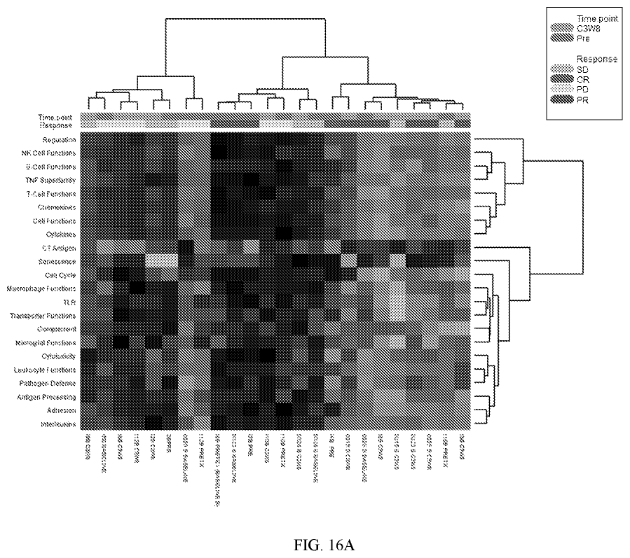

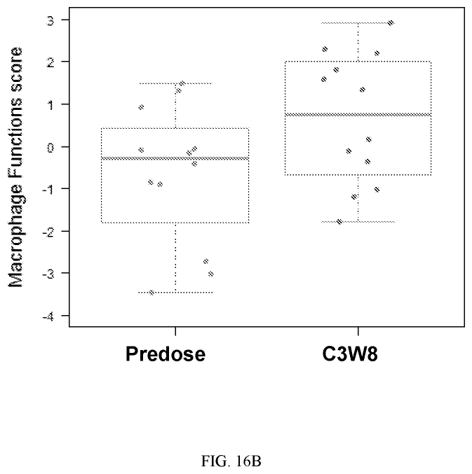

As depicted in FIGS. 9D and 9E, baseline tumor tissue in responding and non-responding patients in the NCT02644967 clinical trial show similar T-cell functional gene signatures and cytotoxic gene signatures. However, intratumoral administration of IMO-2125 shows a significant up regulation of T-cell functional genes (IFNγ, Tbx21, perforin, granzymes) as well as antigen presenting cell activation (CD86, IL12), and genes associated with response to IFNγ (PD-L1, HLA-A, HLA-B, HLA-C) in responding patients at C3W8. Such up regulation was not observed in non-responding patients (n=13, as shown in FIGS. 11A and 11B). Treatment also induced other types of cellular functions, including macrophage function, again by C3W8, and more enriched in responding patients (depicted in FIGS. 16A and 16B).

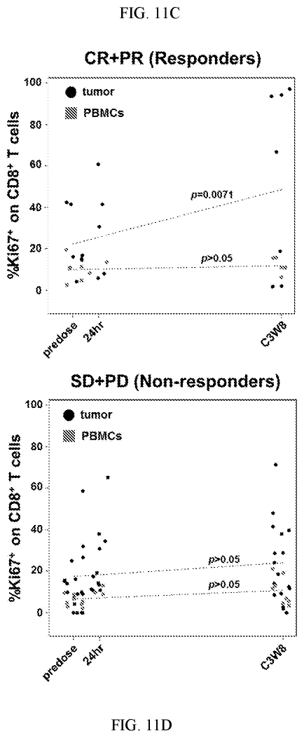

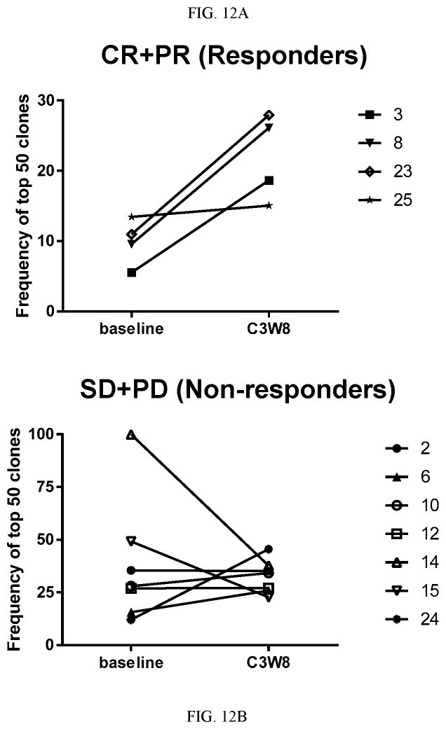

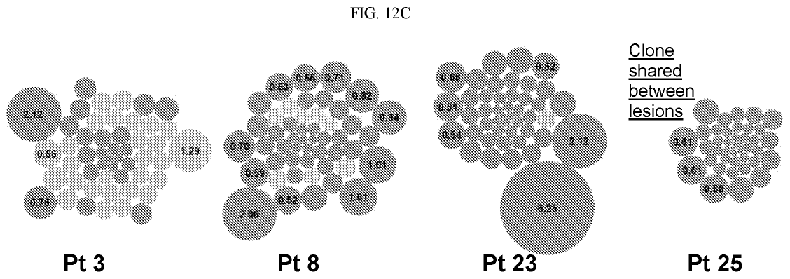

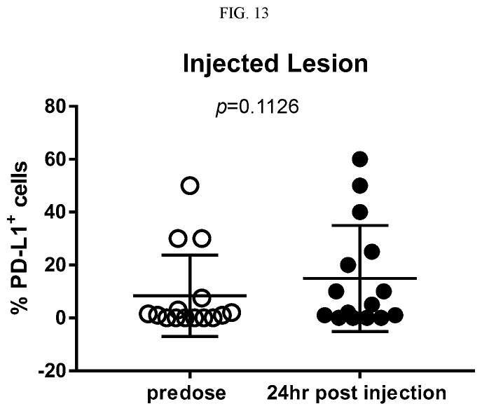

Furthermore, combination therapy drives expansion of the T-cell clones that are shared between intratumoral injected (local) and non-injected (distant) tumors. FIG. 12A shows that such parallel expansion was not observed in those patients that did not respond (that is, patients with stable disease (SD) or progressive disease (PD)). FIG. 12B shows that the comparison between baseline and C3W8 of the local lesion.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. PCT/US17/51742, filed on Sep. 17, 2017, is incorporated by reference in its entirety. U.S. application Ser. No. 15/703,631, filed on Sep. 15, 2017, is incorporated by reference in its entirety.

1. A method for treating a tumor in a patient having low tumor expression of MHC Class I genes, the method comprising:
intratumoral administration of a TLR 9 agonist.

2. The method of claim 1, wherein the TLR agonist has the structure: 5′-TCG1AACG1TTCG1-X-G1CTTG1CAAG1CT-5′ (5′ SEQ ID NO:4-X-SEQ ID NO:4 5′), wherein G1 is 2′-deoxy-7-deazaguanosine and X is a glycerol linker.

3. The method of claim 1, further comprising administration of at least one immune checkpoint inhibitor.

4-5. (canceled)

6. The method of claim 3, wherein the immune checkpoint inhibitor is administered after the TLR9 agonist.

7-8. (canceled)

9. The method of claim 1, wherein the tumor is a metastatic tumor.

10. The method of claim 1, wherein the tumor is selected from melanoma, lung tumor, kidney tumor, prostate tumor, cervical tumor, colorectal tumor, colon tumor, pancreatic tumor, ovarian tumor, urothelial tumor, gastric/GEJ tumor, head and neck tumor, glioblastoma, Merkel cell tumor, head and neck squamous cell carcinoma (HNSCC), non-small cell lung carcinoma (NSCLC), small cell lung tumor (SCLC), or bladder tumor.

11. The method of claim 9, wherein the tumor is metastatic melanoma.

12-13. (canceled)

14. The method of claim 1, wherein the low expression of tumor MHC Class I gene expression is less than 25% of the expression in healthy tissue.

15. (canceled)

16. The method of claim 3, wherein the immune checkpoint inhibitor is selected from a checkpoint inhibitor that targets PD-1, PD-L1, cytotoxic T-lymphocyte-associated protein 4 (CTLA-4), LAG3, B7-H3, B7-H4, KIR, OX40, IgG, IDO-2, CEACAM1, TNFRSF4, BTLA, OX40L, and TIM3.

17-23. (canceled)

24. The method of claim 2, wherein the TLR9 agonist is administered at a dose of from about 1 mg to about 20 mg.

25-26. (canceled)

27. A method for treating a tumor in a patient comprising:
(a) determining MHC Class I gene expression in a tumor sample and
(b) administering a TLR9 agonist if said gene expression is present in less than 50% of the tumor cells.

28. The method of claim 27, wherein the TLR9 agonist has the structure: 5′-TCG1AACG1TTCG1-X-G1CTTG1CAAG1CT-5′ (5′ SEQ ID NO:4-X-SEQ ID NO:4 5′), wherein G1 is 2′-deoxy-7-deazaguanosine and X is a glycerol linker.

29. The method of claim 27, further comprising administering at least one immune checkpoint inhibitor.

30-52. (canceled)

53. A method for treating a tumor in a patient having increased serum PD-L2 levels, the method comprising:
(a) determining the level of PD-L2 levels in said patient; and
(b) administering a TLR9 agonist if said PD-L2 levels in said patient are increased as compared with a control level of PD-L2.

54. The method of claim 53, wherein the increased serum PD-L2 level is between about 750 pg/mL and 5000 pg/mL.

55-58. (canceled)

59. The method of claim 53, wherein the TLR9 agonist has the structure: 5′-TCG1AACG1TTCG1-X-G1CTTG1CAAG1CT-5′ (5′ SEQ ID NO:4-X-SEQ ID NO:4 5′), wherein G1 is 2′-deoxy-7-deazaguanosine and X is a glycerol linker.

60. The method of claim 53, further comprising administering at least one immune checkpoint inhibitor.

61-62. (canceled)

63. The method of claim 60, wherein the immune checkpoint inhibitor is administered after the TLR9 agonist.

64-66. (canceled)

67. The method of claim 53, wherein the tumor is selected from melanoma, lung tumor, kidney tumor, prostate tumor, cervical tumor, colorectal tumor, colon tumor, pancreatic tumor, ovarian tumor, urothelial tumor, gastric/GEJ tumor, head and neck tumor, glioblastoma, Merkel cell tumor, head and neck squamous cell carcinoma (HNSCC), non-small cell lung carcinoma (NSCLC), small cell lung tumor (SCLC), or bladder tumor.

68-78. (canceled)

79. The method of claim 53, wherein the TLR9 agonist is administered at a dose of from about 1 mg to about 20 mg.

80. (canceled)
